(19) United States
(12) Patent Application Publication
(10) Pub. No.: US 2013/0244293 A1
(43) Pub. Date: Sep. 19, 2013

(54) FRACTIONATED EXTRACTIVE PRODUCTS FROM PLANT BIOMASS AND METHODS OF MAKING AND USING SAME

(71) Applicant: BOARD OF TRUSTEES OF MICHIGAN STATE UNIVERSITY, East Lansing, MI (US)

(72) Inventors: Venkatesh Balan, East Lansing, MI (US); Albert Cheh, Bethesda, MD (US); Shishir Chundawat, Madison, WI (US); Bruce E. Dale, Mason, MI (US); Leonardo Sousa, Lansing, MI (US)

(73) Assignee: BOARD OF TRUSTEES OF MICHINGAN STATE UNVERSITY, East Lansing, MI (US)

(21) Appl. No.: 13/886,021

(22) Filed: May 2, 2013

Related U.S. Application Data
(63) Continuation-in-part of application No. 13/642,052, filed on Dec. 17, 2012.
Application No. 13/642,052 is a continuation-in-part of application No. 61/641,666, filed on May 2, 2012.
Application No. 61/641,666 is a continuation-in-part of application No. 61/817,204, filed on Apr. 29, 2013.
Application No. 61/817,204 is a continuation-in-part of application No. 13/835,382, filed on Mar. 15, 2013.
Application No. 13/835,382 is a continuation-in-part of application No. 13/202,011, filed on Aug. 17, 2011.
(60) Provisional application No. 61/325,560, filed on Apr. 19, 2010.
(60) Provisional application No. 61/236,403, filed on Aug. 24, 2009.

(51) Int. Cl. C07G 1/00; C12P 19/14

(52) U.S. Cl. CPC C07G 1/00; C12P 19/14
USPC 435/99; 530/500; 127/43; 422/129

(57) ABSTRACT

A method is provided comprising converting at least a portion of native cellulose I_{β} to cellulose III_{I} by treating plant biomass with liquid ammonia and/or one or more organic solvents to produce a treated plant biomass containing lignin and a lignin fraction; and extracting lignin and/or other plant cell wall components from the lignin fraction to produce a lignin extract capable of being fractionated. In one embodiment, the lignin extract is fractionated into separate components which are useful in a variety of applications.

This application is a continuation-in-part of U.S. patent application Ser. No. 13/642,052, filed on Oct. 18, 2012, which application is a National Stage Filing under 35 U.S.C. 371 from International Application No. PCT/US2011/033079, filed Apr. 19, 2011, and published in English as WO/2011/133571 on Oct. 27, 2011, which application claims the benefit under 35 U.S.C. 119 (e) of U.S. Provisional Application Ser. No. 61/325,560 filed on Apr. 19, 2010, which applications and publications are hereby incorporated by reference in their entireties. This application also claims the benefit under 35 U.S.C. 119(e) of U.S. Provisional Application Ser. No. 61/641,666 filed on May 2, 2012 and U.S. Provisional Application Ser. No. 61/817,204, filed on Apr. 29, 2013 which applications are hereby incorporated by reference herein in their entireties. This application is also a continuation-in-part of U.S. patent application Ser. No. 13/835,382 filed on Mar. 15, 2013, which application is a continuation-in-part of U.S. patent application Ser. No. 13/202,011 filed on Aug. 17, 2011, which application is a U.S. National Stage Filing under 35 U.S.C. 371 from International Application No. PCT/US2010/046525, filed Aug. 24, 2010, and published in English as WO 2011/028543 on Mar. 10, 2011, which claims the benefit under 35 U.S.C. 119 (e) of U.S. Provisional Application Ser. No. 61/236,403 filed on Aug. 24, 2009, which applications and publications are hereby incorporated by reference in their entireties.

### BACKGROUND

Current attempts to produce cellulosic-based biofuel are cost prohibitive and involve a number of steps.

### SUMMARY

In one embodiment, a method is provided which comprises extracting lignin and/or other plant cell wall extractives in an extractive ammonia (EA) treatment using anhydrous liquid ammonia (w/wo additional organic solvents to facilitate extraction) either during the pretreatment process and/or on untreated and/or AFEX™ pretreated biomass and/or on AFEX™ pretreated densified biomass particulates. The cell wall extractives can, in various embodiments, be further fractionated into fractions, such as four fractions, based on differences in solubility in alcohol (e.g., ethanol) and water.

Using a combination of cellulases and hemicellulases or dilute acid, the extracted biomass, which contains residual oligomeric sugars, can be further hydrolyzed to sugars, with the insoluble lignin recovered as a solid phase. If pretreated biomass is hydrolyzed without any extraction, the lignin can instead be recovered from the fermentation broth after fermenting the sugars, while the insoluble lignin can be obtained as a solid stream. The insoluble lignin can be further purified using proteases to remove bound proteins from the biomass as soluble peptides (followed by repeated washing with water and mild surfactants) or via selectively solubilizing the lignin using organic solvents.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic illustration showing biomass pretreatment using gaseous ammonia in packed bed tubular reactor (PB-AFEX™) according to an embodiment.

FIG. 1B is a schematic illustration showing biomass pretreatment using anhydrous liquid ammonia in an extractive ammonia (EA) treatment according to an embodiment.

FIG. 2 is a schematic illustration showing two biomass biorefinery scenarios, namely de-centralized processing (I) and centralized processing (II) according to various embodiments.

FIG. 3 is a schematic illustration showing the fractions (F1-F4) of ammonia-soluble extractives obtained using EA treatment applied to untreated lignocellulosic biomass, AFEX™ pretreated biomass or pretreated densified biomass particulates using a combination of ethanol and water and Fraction F5 obtained after further processing according to various embodiments.

FIG. 4 is a schematic illustration showing two scenarios when hydrolyzing PB-AFEX™ densified biomass particulates, namely extraction using anhydrous ammonia or organic solvent or a combination of both (I) and no extraction (II) according to various embodiments.

FIG. 5 is a flow chart showing an exemplary EA treatment according to an embodiment which produces Fractions F1-F5.

FIG. 6 is an x-ray diffraction pattern of EA treated corn stover according to an embodiment.

FIG. 7 is a bar graph showing enzymatic hydrolysis performance at 6% glucan loading according to an embodiment.

FIG. 8 is a flow chart showing fractionation and lignin mass balance for an EA treatment according to various embodiments.

FIG. 9 show 2D Heteronuclear Single-Quantum Correlation-Nuclear Magnetic Resonance (HSQC-NMR) (in dimethyl sulfoxide-d6 (DMSO-d6)) of fractions F1-F4 obtained in FIG. 5, with components identified in the aliphatic and aromatic regions according to various embodiments.

FIG. 10 are gel permeation chromatography graphs of fractions F1-F5 of FIG. 5 according to various embodiments.

FIG. 11 show elemental analysis graphs of fractions F1-F5 of FIG. 5, including a more detailed elemental analysis graph of F3 (i.e., carbon, hydrogen, nitrogen and oxygen) according to various embodiments.

FIG. 12 is an exemplary mass balance on an EA treatment on a given amount of AFEX™ pretreated biomass according to an embodiment.

### DETAILED DESCRIPTION OF THE EMBODIMENTS

In the following detailed description, embodiments are described in sufficient detail to enable those skilled in the art to practice them, and it is to be understood that other embodiments may be utilized and that chemical and procedural changes may be made without departing from the spirit and scope of the present subject matter. The following detailed description is, therefore, not to be taken in a limiting sense, and the scope of embodiments is defined only by the appended claims.

The term “biomass” as used herein, refers in general to organic matter harvested or collected from a renewable biological resource as a source of energy. The renewable biological resource can include plant materials, animal materials, and/or materials produced biologically. The term “biomass” is not considered to include fossil fuels, which are not renewable.

The term “plant biomass” or “ligno-cellulosic biomass (LCB)” as used herein is intended to refer to virtually any plant-derived organic matter containing cellulose and/or hemicellulose as its primary carbohydrates (woody or non-woody) available for producing energy on a renewable basis. Plant biomass can include, but is not limited to, agricultural residues such as corn stover, wheat straw, rice straw, sugar cane bagasse and the like. Plant biomass further includes, but is not limited to, woody energy crops, wood wastes and residues such as trees, including fruit trees, such as fruit-bearing trees, (e.g., apple trees, orange trees, and the like), softwood forest thinnings, barky wastes, sawdust, paper and pulp industry waste streams, wood fiber, and the like. Additionally perennial grass crops, such as various prairie grasses, including prairie cord grass, switchgrass, Miscanthus, big bluestem, little bluestem, side oats grama, and the like, have potential to be produced large-scale as additional plant biomass sources. For urban areas, potential plant biomass feedstock includes yard waste (e.g., grass clippings, leaves, tree clippings, brush, etc.) and vegetable processing waste. Plant biomass is known to be the most prevalent form of carbohydrate available in nature and corn stover is currently the largest source of readily available plant biomass in the United States. When describing the various embodiments and used without a qualifier, the term “biomass” is intended to refer to “plant biomass,” i.e., lignocellulosic biomass (LCB).

The term “biofuel” as used herein, refers to any renewable solid, liquid or gaseous fuel produced biologically and/or chemically, for example, those derived from biomass. Most biofuels are originally derived from biological processes such as the photosynthesis process and can therefore be considered a solar or chemical energy source. Other biofuels, such as natural polymers (e.g., chitin or certain sources of microbial cellulose), are not synthesized during photosynthesis, but can nonetheless be considered a biofuel because they are biodegradable. There are generally considered to be three types of biofuels derived from biomass synthesized during photosynthesis, namely, agricultural biofuels (defined below), municipal solid waste biofuels (residential and light commercial garbage or refuse, with most of the recyclable materials such as glass and metal removed) and forestry biofuels (e.g., trees, waste or byproduct streams from wood products, wood fiber, pulp and paper industries). Biofuels produced from biomass not synthesized during photosynthesis include, but are not limited to, those derived from chitin, which is a chemically modified form of cellulose known as an N-acetyl glucosamine polymer. Chitin is a significant component of the waste produced by the aquaculture industry because it comprises the shells of seafood.

The term “agricultural biofuel”, as used herein, refers to a biofuel derived from agricultural crops, lignocellulosic crop residues, grain processing facility wastes (e.g., wheat/oat hulls, corn/bean fines, out-of-specification materials, etc.), livestock production facility waste (e.g., manure, carcasses, etc.), livestock processing facility waste (e.g., undesirable parts, cleansing streams, contaminated materials, etc.), food processing facility waste (e.g., separated waste streams such as grease, fat, stems, shells, intermediate process residue, rinse/cleansing streams, etc.), value-added agricultural facility byproducts (e.g., distiller's wet grain (DWG) and syrup from ethanol production facilities, etc.), and the like. Examples of livestock industries include, but are not limited to, beef, pork, turkey, chicken, egg and dairy facilities. Examples of agricultural crops include, but are not limited to, any type of non-woody plant (e.g., cotton), grains such as corn, wheat, soybeans, sorghum, barley, oats, rye, and the like, herbs (e.g., peanuts), short rotation herbaceous crops such as switchgrass, alfalfa, and so forth.

The term “pretreatment step” as used herein, refers to any step intended to alter native biomass so it can be more efficiently and economically converted to reactive intermediate chemical compounds such as sugars, organic acids, etc., which can then be further processed to a variety of end products such as ethanol, isobutanol, long chain alkanes etc. Pretreatment can reduce the degree of crystallinity of a polymeric substrate, reduce the interference of lignin with biomass conversion by hydrolyzing some of the structural carbohydrates, thus increasing their enzymatic digestibility and accelerating the degradation of biomass to useful products. Pretreatment methods can utilize acids of varying concentrations, including dilute acid pretreatments, concentrated acid pretreatments (using, for example, sulfuric acids, hydrochloric acids, organic acids, and the like) and/or alkali such as ammonia and/or ammonium hydroxide and/or calcium hydroxide and/or sodium hydroxide and/or lime, and the like. Pretreatment methods can additionally or alternatively utilize hydrothermal treatments including water, heat, steam or pressurized steam pretreatments, including, but not limited to, hydro-thermolysis pretreatment and liquid hot water pretreatment, further including, for example, acid catalyzed steam explosion pretreatment. Pretreatment can occur or be deployed in various types of containers, reactors, pipes, flow through cells and the like. Most pretreatment methods will cause the partial or full solubilization and/or destabilization of lignin and/or hydrolysis of hemicellulose to pentose sugars. Further examples of pretreatment include, but are not limited wet oxidation, organosolv pretreatment and mechanical extrusion.

The term “moisture content” as used herein, refers to percent moisture of biomass. The moisture content is calculated as grams of liquid, such as water per gram of wet biomass (biomass dry matter plus liquid times 100%. As such, when used without qualification herein, the % moisture content refers to a total weight basis.

The term “Ammonia Fiber Expansion” (hereinafter “AFEX™”) pretreatment” as used herein, refers to a process for pretreating biomass with liquid and/or vapor (gaseous) ammonia to solubilize lignin from plant cell walls and redeposit it from in between plant cell walls to the surface of the biomass. An AFEX™ pretreatment disrupts the lignocellulosic matrix, thus modifying the structure of lignin, partially hydrolyzing hemicellulose, and increasing the accessibility of cellulose and the remaining hemicellulose to subsequent enzymatic degradation. Lignin is the primary impediment to enzymatic hydrolysis of native biomass, and removal, relocation or transformation of lignin is a suspected mechanism of several of the leading pretreatment technologies, including AFEX™.

However, in contrast to many other pretreatments, the lower temperatures and non-acidic conditions of the AFEX™ process prevents lignin and/or sugars from being converted into furfural, hydroxymethyl furfural, and organic acids that could negatively affect microbial activity. The AFEX™ process further expands and swells cellulose fibers and further breaks up amorphous hemi-cellulose in lignocellulosic biomass (LCB). These structural changes open up the plant cell wall structure enabling more efficient and complete conversion of LCB to value-added products, while preserving the nutrient value and composition of the material. In one embodiment, the EA treatment serves as a pretreatment.

The term “gaseous AFEX™ pretreatment” or “GAP™” as used herein, refers to an AFEX™ pretreatment as defined herein, which uses gaseous ammonia rather than liquid ammonia. By allowing hot ammonia gas, i.e., ammonia vapors (with or without a carrier) to condense directly on cooler biomass, the biomass heats up quickly and the ammonia and biomass come into intimate contact.

The term “extractive ammonia treatment” or “EA” as used herein, refers to a process that removes extractives (i.e., biomass degraded products and plant metabolites) during anhydrous liquid ammonia based biomass treatment with or without an organic solvent. EA treatment can also be used as a pretreatment.

The term “added binder” as used herein, refers to natural and/or synthetic substances and/or energy forms added or applied to biomass in an amount sufficient to improve stability of a densified biomass particulate. Examples of commonly added binders include, but are not limited to, exogenous heat, steam, water, air, corn starch, lignin compounds, lignite, coffee grounds, sap, pitch, polymers, salts, acids, bases, molasses, organic compounds, urea, and tar. Specialty additives are also used to improve binding and other pellet properties such as color, taste, pH stability, and water resistance. Added binder in the form of added energy is typically in the form of heat which is added outright, i.e., exogenous heat, such as convective or conducted heat, although radiated heat may also be used for the same purpose. The intentional addition of exogenous heat is in contrast to intrinsic heat which develops as a result of a material being processed, such as the heat of friction which develops in densification equipment during operation. As such, heat which is inherent to the pretreatment and/or densification of biomass is not considered herein to be “added binder.” Added binder may be added to the pretreated biomass at any time before, during or after a densification process. The amount of added binder can vary depending on the substrate being densified.

The term “particulate” or “biomass particulate” as used herein refers to densified (i.e., solid) biomass formed from a plurality of loose (which can include “chopped”) biomass fibers which are compressed to form a single particulate product which is dividable into separate pieces. A particulate can be hydrolysable or non-hydrolysable and can range in size from small microscopic particles (larger than powders) to pellets or large objects, such as bricks, or larger, such as hay bales or larger, with any suitable mass. The specific geometry and mass will depend on a variety of factors including the type of biomass used, the amount of compression used to create the particulate, the desired length of the particulate, and the particular end use.

The term “briquette” as used herein refers to a compressed particulate.

The term “pellet” as used herein refers to an extruded particulate, i.e., a compressed particulate formed with a shaping process in which material is forced through a die.

The term “bulk density” as used herein, refers to the mass or dry weight of a quantity of particles or particulates (granules and other “divided” solids) divided by the total volume they occupy (mass/volume). Therefore, bulk density is not an intrinsic property of the particles, as it is changeable when the particles are subjected to movement from an external source. The volume measurement is a combination of the particle volume (which includes the internal pore volume of a particle) and the intra-particle void volume. Bulk density=intrinsic density (of each particle)×(1−voids fraction). For a given intrinsic particle density, therefore, the bulk density depends only on the void fraction, which is variable.

The term “moisture content” as used herein, refers to percent moisture of biomass. Moisture content can be expressed on a dry weight basis (dwb) or a total weight basis, i.e., moisture wet basis (mwb). The total moisture content is calculated as grams of liquid, such as water per gram of wet biomass (biomass dry matter plus water) liquid times 100%. However, when used without qualification herein, the % moisture content refers to a dry weight basis.

The term “flowability” as used herein refers to the ability of particulates to flow out of a container using only the force of gravity. A product having increased flowability, therefore, would flow out of the container at a faster rate as compared to a product having lower flowability.

The term “logistical properties” as used herein refers to one or more properties of a particulate related to storage, handling, and transportation, which can include, but are not limited to stability, shelf life, flowability, high bulk density, high true density, compressibility, durability, relaxation, springback, permeability, unconfined yield strength, and the like.

Nearly all forms of lignocellulosic biomass, i.e., plant biomass, such as monocots, comprise three primary chemical fractions: hemicellulose, cellulose, and lignin. Hemicellulose is a polymer of short, highly-branched chains of mostly five-carbon pentose sugars (xylose and arabinose), and to a lesser extent six-carbon hexose sugars (galactose, glucose and mannose). Dicots, on the other hand, have a high content of pectate and/or pectin, which is a polymer of alpha-linked glucuronic acid. Pectate may be “decorated” with mannose or rhamnose sugars, also). These sugars are highly substituted with acetic acid.

Because of its branched structure, hemicellulose is amorphous and relatively easy to hydrolyze (breakdown or cleave) to its individual constituent sugars by enzyme or dilute acid treatment. Cellulose is a linear polymer of glucose sugars, much like starch, which is the primary substrate of corn grain in dry grain and wet mill ethanol plants. However, unlike starch, the glucose sugars of cellulose are strung together by 13-glycosidic linkages which allow cellulose to form closely-associated linear chains. Because of the high degree of hydrogen bonding that can occur between cellulose chains, cellulose forms a rigid crystalline structure that is highly stable and much more resistant to hydrolysis by chemical or enzymatic attack than starch or hemicellulose polymers. Lignin, which is a polymer of phenolic molecules, provides structural integrity to plants, and remains as residual material after the sugars in plant biomass have been fermented to ethanol. Lignin is a by-product of alcohol production and is considered a premium quality solid fuel because of its zero sulfur content and heating value, which is near that of sub-bituminous coal.

Typically, cellulose makes up 30 to 50% of residues from agricultural, municipal, and forestry sources. While cellulose is more difficult to convert to ethanol than hemicellulose, it is the sugar polymers of hemicellulose which can be more readily hydrolyzed to their individual component sugars for subsequent fermentation to ethanol. Although hemicellulose sugars represent the “low-hanging” fruit for conversion to ethanol, the substantially higher content of cellulose represents the greater potential for maximizing alcohol yields, such as ethanol, on a per ton basis of plant biomass.

As noted above, the hemicellulose fraction of biomass contains hexose and pentose sugars, while the cellulose fraction contains glucose. In current AFEX™ pretreatment operations, only limited hemicellulose conversions are obtained. It is further known that of the sugars extracted, about 30 to 35% is xylose and about 35 to 40% is glucose (most all of which is currently converted only in post-pretreatment steps). Overall conversions, as well as over-all ethanol yields, will vary depending on several factors such as biomass type, pretreatment type, and so forth.

Conventional methods used to convert biomass to alcohol include processes employing, for example, a concentrated acid hydrolysis pretreatment, a two-stage acid hydrolysis pretreatment as well as processes employing any known conventional pretreatment, such as hydrothermal or chemical pretreatments, followed by an enzymatic hydrolysis (i.e., enzyme-catalyzed hydrolysis) or simultaneous enzymatic hydrolysis and saccharification. Such pretreatment methods can include, but are not limited to, dilute acid hydrolysis, high pressure hot water-based methods, i.e., hydrothermal treatments such as steam explosion and aqueous hot water extraction, reactor systems (e.g., batch, continuous flow, counter-flow, flow-through, and the like), AFEX™, ammonia recycled percolation (ARP), lime treatment and a pH-based treatment.

Several of these methods generate nearly complete hydrolysis of the hemicellulose fraction to efficiently recover high yields of the soluble pentose sugars. This also facilitates the physical removal of the surrounding hemicellulose and lignin, thus exposing the cellulose to later processing. However, most, if not all, pretreatment approaches do not significantly hydrolyze the cellulose fraction of biomass.

In one embodiment, an ammonia fiber expansion method (AFEX™) pretreatment is used as defined herein. As such, the AFEX™ pretreatment can be a liquid and/or gaseous ammonia pretreatment, further including a packed bed (PB)-AFEX™ pretreatment as defined herein. Such treatments can be performed according to the methods described in, for example, U.S. Pat. Nos. 6,106,888 ('888), 7,187,176 ('176), 5,037,663 ('663), and 4,600,590 ('590) and 8,394,691 ('691), each of which is hereby incorporated by reference in its entirety. See also, for example, U.S. Pat. No. 7,901,517, U.S. patent application Ser. Nos. 13/591,092, 12/976,344, and PCT Publication No. WO/2012/088429, each of which is hereby incorporated by reference herein in its entirety. See also U.S. Pat. No. 7,937,851, U.S. patent application Ser. Nos. 12/791,703 and 13/458,568, and PCT Publication No. WO/2011/153128, each of which is incorporated by reference herein in its entirety. In various embodiments steam and/or water is also used during pretreatment.

In one embodiment, biomass is heated to a temperature of from about 60° C. to about 100° C. or higher, such as up to about 150° C., including any range or value therebetween, in the presence of non-volatile base, such as ammonia (e.g., concentrated ammonia) and/or various hydroxide bases, such as calcium hydroxide, sodium hydroxide, and the like. See, for example, Dale, B. E. et al., 2004, Pretreatment of corn stover using ammonia fiber expansion (AFEX™), Applied Biochem, Biotechnol. 115: 951-963, which is incorporated herein by reference in its entirety. A rapid pressure drop then causes a physical disruption of the biomass structure, exposing cellulose and hemicellulose fibers, without the extreme sugar degradation common to many pretreatments.

In one embodiment, nearly all of the ammonia can be recovered and reused while the remaining ammonia serves as a nitrogen source for microbes in fermentation. In one embodiment, about one (1) to about two (2) wt % of the non-volatile base, such as ammonia, remains on the pretreated biomass.

Additionally, since there is no wash stream in the process, dry matter recovery following an AFEX™ pretreatment is essentially quantitative. This is because AFEX™ pretreatment is basically a dry to dry process.

AFEX™ pretreated biomass is also stable for longer periods (e.g., up to at least a year) than non-AFEX™ pretreated biomass and can be fed at very high solids loadings (such as at least about 40%) in enzymatic hydrolysis or fermentation process as compared with dilute acid or other aqueous pretreatments that cannot easily exceed 20% solids.

Cellulose and hemicellulose are also well-preserved in an AFEX™ pretreatment, showing little degradation. As such, there is no need for neutralization prior to enzymatic hydrolysis of AFEX™ pretreated biomass. Enzymatic hydrolysis of AFEX™ pretreated biomass also produces clean sugar streams for subsequent fermentation.

Degradation products from AFEX™ pretreated biomass have also been identified and quantified. One such study compared AFEX™ pretreated and acid pretreated corn stover using LC-MS/GC-MS techniques. In acid-pretreated feedstock, over 40 major compounds were detected, including organic acids, furans, aromatic compounds, phenolics, amides and oligosaccharides. AFEX™ pretreatment performed under mild alkaline condition produced very little acetic acid, HMF, and furfural. See, for example, Dale, B. E. et al., 2004, supra, and Dale, B. E. et al, 2005b, Pretreatment of Switchgrass Using Ammonia Fiber Expansion (AFEX™), Applied Biochemistry and Biotechnology. Vol. 121-124. pp. 1133-1142. See also Dale, B. E. et al., 2005a. Optimization of the Ammonia Fiber Explosion (AFEX) Treatment Parameters for Enzymatic Hydrolysis of Corn Stover, Bioresource Technology. Vol. 96, pp. 2014-2018 and Chundawat, et al., 2010, Multifaceted characterization of cell wall decomposition products formed during ammonia fiber expansion (AFEX) and dilute-acid based pretreatments, Bioresource Technology Vol. 101, pp. 8429-8438, each of which is incorporated herein in its entirety.

As noted above, the main components of lignocellulosic biomass (or plant cell walls, which contribute to the majority of the biomass by percent weight) are cellulose, hemicelluloses, lignin, protein and ash. The ratio of these components varies among families of plants, like grasses, softwoods and hardwoods. In angiosperms, the lignin polymer is produced by dehydrogenative polymerization of three different cinnamyl alcohols (p-coumaryl, coniferyl, and sinapyl alcohol) that differ in the degree of methoxylation at the C3 and C5 positions of the aromatic ring. When these alcohols are transformed in lignin polymers, they are called p-hydroxyphenyl (H), guaiacyl (G) and syringyl (S) units of the polymer, respectively.

Lignin's complexity and heterogeneity depends on the relative proportions of the three main monolignol units (H, G and S). Lignin from grasses incorporates G and S units at comparable levels and more H units than hard woods. In softwoods, lignin is essentially made of G units while in hardwoods it is made of G and S units. Lignin rich in G units has relatively more carbon-carbon bonds than lignin rich in S units. In addition to these three main components, lignin polymers incorporate compounds that derive from incomplete biosynthesis of components H, G and S, as well as other phenylpropanoid units such as acetates, hydroxycinnamyl aldehydes and others (Sederoff et al. 1999). Native lignin in plants is highly cross-linked with molecular weight >10,000 Da.

Lignin is useful for a variety of applications including, but not limited to, carbon fiber composites, bio-oil, resins, adhesive binders and coating, plastics, paints, enriching soil organic carbon, fertilizer, rubbers and elastomers, paints, antimicrobial agents and slow nitrogen release fertilizer, and the like, and can be a substitute for polymers produced using crude oil.

One current source of lignin in the market is produced from sulfite (or sulfonate) based paper/pulp mills. Most such mills currently burn the lignin to recover energy, in an attempt to reduce the environmental impact of discharge. Very few sulfite mills currently process the lignosulfonates from sulfite spent liquors. Additionally, the quality and quantity of lignin obtained via currently known methods are inadequate for most applications.

Thus, there is a need for reliable high quality processed lignin having more reactive groups with good dispersing, binding, emulsifying, water repellent and conductive properties. The various embodiments described herein can provide lignin having at least these features. Furthermore, most applications for lignin require low ash and carbohydrate contents. As such, in one embodiment, the extractives produced with the methods described herein are fractionated into first two, and then four fractions to obtain even cleaner lignin fractions. This novel approach relies on the difference in solubility, with precipitation and filtration-based methods being utilized to obtain the distinct fractions described herein.

In various embodiments, methods of extracting lignin and other plant cell wall extractives are provided, using anhydrous liquid ammonia (w/wo additional organic solvents to facilitate extraction) during and/or after a pretreatment process (e.g., ammonia pretreatment, such as AFEX™) of loose biomass fibers, from the resulting pretreated hydrolysable loose biomass fibers or from pretreated hydrolysable densified biomass particulates (hereinafter “hydrolysable particulates”), such as pellets, briquettes, and the like. In various embodiments, such extraction can be performed on loose biomass fibers or densified biomass particulates which have not been pretreated and which therefore have reduced hydrolysable properties as compared with their pretreated counterparts. The cell wall extractives can further be fractionated into four major fractions based on differences in solubility in alcohol (e.g., ethanol) and water.

See, for example, U.S. patent application Ser. Nos. 13/458,830 (hereinafter application “'830), as well as U.S. patent application Ser. Nos. 13/835,382, 13/835,766 and 13/202,011, each of which is hereby incorporated herein by reference in its entirety. Application '830, for example, discusses methods for pretreating and densifying loose biomass fibers to produce hydrolysable particulates.

In contrast to conventional densification processes, the embodiments described therein do not rely on added binder for improving the logistical properties or stability of the resulting hydrolysable particulates. Rather, and as discussed therein, hydrolysable particulates are produced without adding binder, i.e., with “no added binder,” during the densification stage, and, in various embodiments, without adding or applying added binder before densification or after densification. Of note, since lignin is generally darker than other components in plant material, the resulting particulates are noticeably darker in appearance than a material not substantially surrounded by lignin.

In one embodiment, ammonia pretreatment is used to pretreat biomass. Since ammonia is a volatile alkali, it can be recovered and re-used. In one embodiment, there are substantially no water wash streams or waste generated using an AFEX™ pretreatment. The resulting product can be, in various embodiments, an almost dry pretreated biomass (8-15% of moisture content). Conventional AFEX™ pretreatment can be performed, for example, using liquid ammonia (about 0.3 to about 2 g NH₃/g dry biomass) with moist biomass (i.e., about 0.1 to about 2 g H₂O/g dry biomass). The batch process involves heating the biomass-water-ammonia mixture (to about 40 to about 180° C.) for a desired period of time (e.g., about 5 to about 60 min) before rapidly releasing the pressure (See Balan et. al., 2009).

Variations in the process have revolved around the manner in which the ammonia comes into contact with the biomass. For example, EA treatment, as defined herein, refers to a process in which the ammonia contacts the biomass in a liquid state. The term Gaseous Ammonia Pretreatment or GAP™, as defined herein, refers to a process in which the ammonia contacts the biomass as a vapor, which then condenses onto the biomass. Other variations of the conventional AFEX™ pretreatment process involve varying moisture contents of the biomass, and/or the method of ammonia recovery and/or re use following pretreatment.

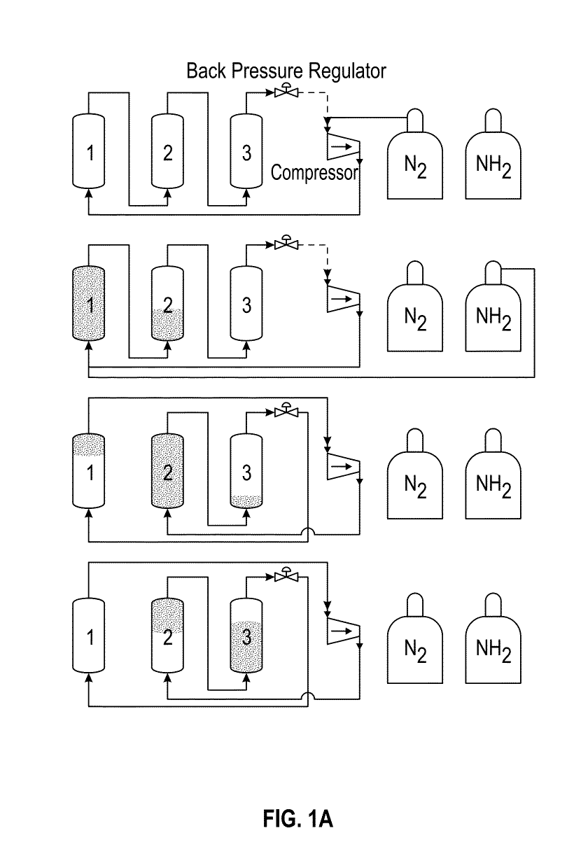

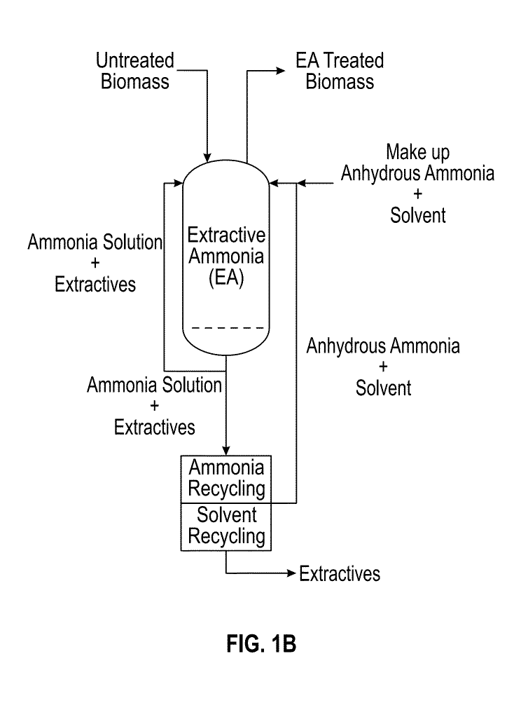

In one embodiment, the AFEX™ pretreatment is PB-AFEX™. See, for example, U.S. Pat. Nos. 8,394,177 and 7,937,851, and U.S. patent application Ser. No. 13/458,568, each of which is hereby incorporated herein by reference in its entirety. In PB-AFEX™, gaseous ammonia is used to pretreat biomass packed in a bed reactor. After pretreatment, the ammonia gas is transferred to the second packed bed reactor. The residual ammonia in the pretreated biomass in a first bed is pushed out by steam to the second reactor (See, for example, FIG. 1A). The process can proceed as a continuous cycle, with recycle of ammonia repeatedly followed by treatment of biomass, as shown in FIG. 1A

EA treatment, shown in FIG. 1B, is based on the utilization of concentrated anhydrous ammonia in high liquid-to-solid ratios, enabling the conversion of native cellulose I to cellulose III allomorph, as well as the extraction of biomass components (e.g. lignin and hemicelluloses). As is understood by those skilled in the art, non-native cellulose III requires lesser enzymes during enzyme hydrolysis and is considerably more digestible than cellulose I).

See, for example, Chundawat, S. P. S. et al., Restructuring the crystalline cellulose hydrogen bond network enhances its depolymerization rate. Journal of American Chemical Society 133 (29), 11163-11174 (2011); Chundawat, S. P. S. et al., Multifaceted characterization of cell wall decomposition products formed during ammonia fiber expansion (AFEX) and dilute-acid based pretreatments. Biores Technol 101, 8429-8438 (2010); Chundawat, S. P. S. et al., Multi-scale visualization and characterization of plant cell wall deconstruction during thermochemical pretreatment. Energy & Environmental Science 4 (3), 973-984 (2011); and Chundawat, S. P. S. et al., Restructuring the crystalline cellulose hydrogen bond network enhances its de-polymerization rate. Journal of American Chemical Society 133 (29), 11163-11174 (2011), each of which is hereby incorporated by reference herein in its entirety.

The EA plant cell wall treatment and fractionation can be carried out simultaneously in a single stage process or separately in multistage processes. The parameters controlled during each stage encompass temperature, residence time, pressure, co-solvent type and mixture composition, and ammonia loading.

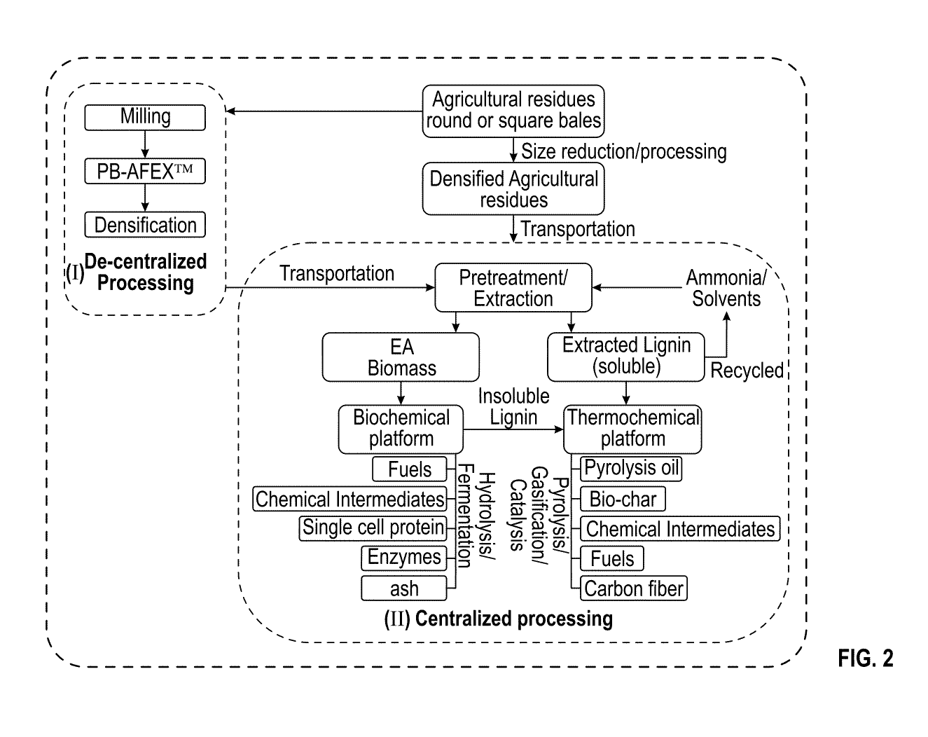

Processing and converting biomass to biofuels can be accomplished with either (I) decentralized processing, or (II) centralized processing, as shown in FIG. 2. Depending on how the process is performed, lignin can end up in different streams. For example, in the decentralized processing (I) of FIG. 2 lignin would not be extracted prior to hydrolysis and fermentation (e.g., either separate hydrolysis and fermentation or simultaneous saccharification and co-fermentation). As such, soluble lignin is present in the sugar stream and accumulates in the stillage stream after a distillation step.

In the decentralized processing (I) shown in FIG. 2, biomass can be harvested and baled (round or square) using conventional procedures and then transported to Local Biomass Processing Depots or Centers (LBPDs or LBPCs). The biomass can be processed and densified at the farm using established techniques (mechanical densification technology or Agglomeration) (Thumuluru et. al., 2011) and then transported to the biorefinery.

In one embodiment, LBPDs are sized at 100-200 tons/day and accept biomass from roughly a 16 km (10 mile) radius. Approximately 5-10 LBPDs can supply feedstock to a single, centralized biorefinery 40 million gal/year (1.514×10⁸ L/year) capacity. The biomass is then ground, pretreated using a pretreatment, such as PB-AFEX™, densified, and transported to central biorefinery.

With the centralized processing scenario (II) shown in FIG. 2, the pretreatment can be perform using any suitable pretreatment process, such as, for example, PB-AFEX™. Readily soluble lignin present within PB-AFEX™ densified or non-densified biomass can then be removed using ammonium hydroxide/liquid ammonia, solvents, or a combination thereof. By using concentrated or anhydrous liquid ammonia (3-6 times the volume of the biomass), PB-AFEX™ biomass containing cellulose I can be activated to cellulose III even at low temperatures (i.e., less than about 25° C.) at very low pressures (less than about 150 psi (10.2 atm)). Therefore, performing a low temperature EA treatment at a centralized biorefinery using PB-AFEX™ pretreated biomass can provide a significant cost reduction while simultaneously allowing for extraction of lignin and activate cellulose I to III.

Ammonia cleaves ester linkages, not ether linkages, during pretreatment and therefore produces more homogeneous high molecular weight lignin as compared to a conventional pulping process. Unlike other lignin extraction processes (where the catalyst is solubilized along with lignin and thus relies on expensive processing steps to recover the catalyst), ammonia is a volatile alkali that can be easily recovered. In one embodiment, more than about 97% of the ammonia is recovered with the remaining approximately 3% having reacted with cell wall components. This ammonia can be re-used during pretreatment, thus leaving behind a relatively clean lignin stream.

Plant cell walls also have a significant distribution in the molecular weight range and degree of cross linking for lignin. Therefore, in one embodiment, it is expected that a significant fraction of lignin that is not extensively cross-linked within the cell wall can be recovered. In one embodiment, liquid ammonia e.g., anhydrous ammonia is used in combination with organic solvents, (i.e., an organosolv process) for extracting and solubilizing a significant fraction of the native lignin, which, in one embodiment, can then be further purified (e.g., by removing ash, oligosaccharides, protein and other biomass components) based on differences in solubility using inexpensive solvents like water and ethanol.

Upgrading lignin through solvent fractionation is expected to yield higher value products that can be used for a variety of applications. The insoluble lignin fraction left behind in the cell walls along with cellulose and hemicelluloses can, in one embodiment, be recovered after enzymatic hydrolysis (or fermentation).

Since polysaccharide degrading enzymes can de-polymerize sugar polymers into soluble sugars (glucose, xylose, arabinose, mannose and galactose, gluco-oligosaccharides and xylo-oligosaccharides), these components can also be easily separated by mechanical separation (e.g., centrifugal decantation) from insoluble biomass residue abundant in lignin. The insoluble lignin rich residue after hydrolysis can, in one embodiment, be further processed using proteases to remove bound enzymes and plant proteins to smaller water soluble peptides (that can be removed by repeated washing with water and mild surfactants) leaving behind a residue rich in insoluble and higher molecular weight lignin.

In one embodiment, the extraction can be carried out either on untreated biomass or biomass that has been prior pretreated using any suitable pretreatment methods, such as a PB-AFEX™ pretreatment. For extraction of lignin from untreated biomass, in one embodiment, a single stage EA treatment can be carried out at high thermochemical severities (high temperature, high pressures). In one embodiment, extraction of lignin from PB-AFEX™ treated biomass is carried out using a mild EA treatment at low temperatures (i.e., less than about 50° C.) and pressures (i.e., less than about 20 atm).

In one embodiment, a method is provided comprising converting at least a portion up to substantially all native cellulose I_{β} to cellulose III_{I} by treating plant biomass with liquid ammonia and/or one or more organic solvents to produce a treated plant biomass containing lignin and a lignin fraction; and extracting lignin and/or other plant cell wall components (e.g., hemicellulose, arabinan, and combinations and degradation products thereof) from the lignin fraction to produce a lignin extract capable of being fractionated.

In one embodiment, β-aryl ether bonds in the lignin are preserved. In one embodiment, the other cell wall components are selected from hemicellulose, arabinan, and combinations and degradation products thereof.

In one embodiment, the plant biomass is a monocot or dicot.

In one embodiment, the extracting step (i.e., Extractive Ammonia Treatment) is a pretreatment step and the plant biomass comprises untreated plant biomass (densified biomass particulates or loose biomass). In one embodiment, the extracting step is performed simultaneously with a pretreatment step. In one embodiment, the pretreatment step comprises liquid ammonia fiber expansion (AFEX) pretreatment or gaseous AFEX pretreatment, e.g., a packed bed-AFEX (PB-AFEX) pretreatment step. In one embodiment, the plant biomass comprises one or more densified biomass particulates.

In one embodiment, the liquid ammonia is selected from anhydrous ammonia (greater than 99.99% ammonia), concentrated ammonia (about 30 to about 99.99%), dilute ammonium hydroxide (less than 30%) in concentration with respect to the liquid present, e.g., water, either inherently present in the biomass or added separately, including separately from the ammonium hydroxide (See, for example, U.S. Pat. No. 8,394,611). Any combination of liquid ammonia can also be provided and/or added at any suitable time.

In one embodiment, the one or more organic solvents comprise any suitable solvent or combination of solvents, including, but not limited to, alcohols (e.g., ethanol), ketones (e.g., acetone), esters (e.g., ethyl acetate), and/or aromatics (e.g., toluene), and the like. Any suitable amount of organic solvent can be added to the ammonia (if present), ranging from trace amounts (˜0% w/w of final solvent composition) up to substantially all organic solvent (˜99.99% w/w of final solvent composition), including any range there between.

In one embodiment, the method further comprises fractionating at least a portion of the lignin extract into four individual fractions based on differences in solubility in one or more polar or non-polar solvents and water.

In one embodiment, the fractions comprise an alcohol insoluble/water soluble fraction (F1), an alcohol insoluble/water insoluble fraction (F2), an alcohol soluble and water insoluble fraction (F3) and an alcohol soluble and water soluble fraction (F4). In one embodiment, the alcohol is ethanol.

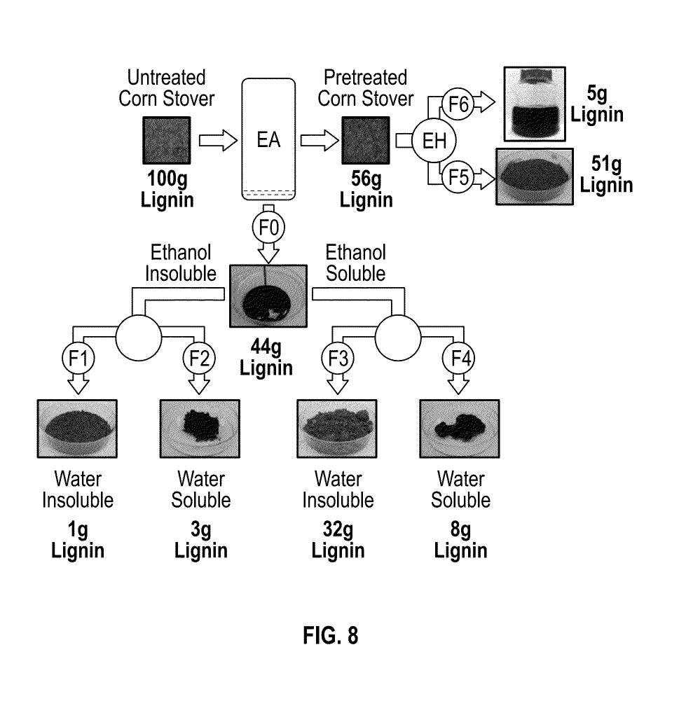

In one embodiment, the treated plant biomass is hydrolyzed (such as with enzymatic hydrolysis) to produce an insoluble lignin fraction (F5) and a hydrolyzed monomeric sugar fraction containing lignin (F6). (See FIG. 8). In various embodiments F5 can be further processed to produce substantially pure lignin. In various embodiments, the further processing can comprise use of suitable enzymes (e.g., proteases), a mild surfactant as understood by those skilled in the art (e.g., Tween®) and water.

In one embodiment, at least 30%, such as at least 40%, such as at least 50% or between about 30 and about 50%, including any range therebetween of the lignin present in the biomass is recovered. In one embodiment, between about 40 and about 50% is recovered, such as between about 42 and about 48%, such as at least about 44% is recovered. In one embodiment, the biomass is corn stover. In one embodiment, lignin is extracted under mild conditions, including a temperature no greater than about 120° C. and a residence time of no more than about 30 min. In one embodiment, the ammonia loading to biomass ratio can be between about 3:1 and 6:1 or no less or no more than about 6:1.

The fractionated lignin can be used as a starting material for several applications, including, but not limited to, carbon fiber composites, bio-oil, resins, adhesive binders and coating, plastics, paints, enriching soil organic carbon, fertilizer, rubbers and elastomers, paints, antimicrobial agents and slow nitrogen release fertilizer (extracted lignin which has been ammoniated).

In one embodiment, F1 comprises primarily minerals and is useful as a fertilizer.

In one embodiment, F2 comprises primarily oligomeric sugars and is also useful as a fertilizer. In one embodiment, the oligomeric sugars in F2 can be separated and used as enzyme inducers.

F3 contains, in one embodiment, at least 90%, such as about at least 92% lignin. In one embodiment, between about 30 and about 35% of the lignin, such as between about 31 and about 34%, such as at least 32% of the lignin, such as up to 32% of the lignin extracted from the plant biomass is from fraction F3.

In one embodiment, F3 contains mostly low molecular weight lignin components which are useful as antimicrobial agents in paints, adhesive binders and coatings, and the like. In one embodiment, the low molecular weight lignin components can be further reduced by catalytic hydrogenation.

In one embodiment, F3 has a low mineral content (i.e., less than 0.18 wt %) and, as noted herein, shows potential for further processing of lignin to biofuels and biochemicals through catalytic conversion using known catalysts.

In one embodiment, F4 has aliphatic and aromatic acids (acetic acid, acetamide, ferulic acid, ferulamide, coumaric acid) and coumaryl amide. In one embodiment, F4 contains primarily lignin degraded products and some oligomeric sugars, which may be useful as a polymer precursor. It is also likely that ferulic and coumaric contents are majorly present in F4.

In one embodiment, aliphatic —OH groups are the most abundant in all the fractions. In one embodiment, F4 contains the highest density of —OH groups while F5 contains the lowest.

In one embodiment, F5 is produced in mass quantities, such as up to 100 kg/200 kg of biomass. In one embodiment, F5 is pyrolyzed by fast or hydrothermal pyrolysis to produce bio-oil and biochar. In one embodiment, F5 is burned to supply energy for a biorefinery. F5 contains high molecular weight lignin. In one embodiment, F5 is further purified, such as with dilute acid/surfactant/protease etc., for use as a carbon fiber production or energy generation. In one embodiment, F5 or the lignin present in F5 is useful for making biocomposites (e.g., computer mother boards, panels, and the like) and bio-material applications.

In various embodiments, a product is provided produced according to any of the methods described herein.

In one embodiment, a method is provided comprising converting native cellulose I_{β} to cellulose III_{I} by treating plant biomass with liquid ammonia and/or one or more organic solvents to generate a treated plant biomass; hydrolyzing the treated plant biomass to produce hydrolyzed treated plant biomass; and fermenting the hydrolyzed treated plant biomass to produce a fermentation broth, wherein insoluble lignin present in the fermentation broth is recovered. In one embodiment, the insoluble lignin is further purified to remove bound proteins.

In one embodiment, a reactor system is provided comprising a reactor adapted to convert native cellulose I_{β} to cellulose III_{I} by treating plant biomass with liquid ammonia and/or one or more organic solvents to produce a treated plant biomass containing lignin and a lignin fraction and extract lignin and/or other plant cell wall components from the lignin fraction to produce a lignin extract; and a monitoring system to monitor the reactor.

In one embodiment, the EA treatment is performed in a reactor system comprising up to three (or more) substantially parallel tubular reactor which can be operated independently under varying pressures. In one embodiment, the reactors have at least an approximately 1 L volume and operate interdependently up to pressures of about 1500 psi (102.1 atm).

In one embodiment, a tubular 1 L collection vessel (at the bottom) is used to collect the extractive from three reactors (located at the top). The process begins by adding biomass at an appropriate moisture level into the reactor and sealing the reactor. In one embodiment, leak testing is performed by passing a gas, such as nitrogen under pressure, such as about 500 psi (34 atm). The test gas can then be released and a desired amount of pretreatment components, e.g., liquid ammonia, loaded into the reactor with the biomass and the reactor heated externally by any suitable means, such as, for example,

Once the desired temperature is reached, the reaction can be continued for a desired residence time. The extractive can then be collected at the bottom of the reactor (with similar pressure at the top reactor) through any suitable means, such as gravity separation. After the extractive is collected, the gas remaining following pretreatment, e.g., ammonia, can be released, such as with a needle valve. After completion of the extraction cycle, both extracted biomass and extractive with residual ammonia can be removed by opening the reactor. In one embodiment, the reactor is kept under a hood at least overnight to allow residual gas, e.g., ammonia, to escape. The gas can then be stored in an appropriate container and reused.

The various embodiments will be further described by reference to the following examples, which are offered to further illustrate various embodiments. It should be understood, however, that many variations and modifications may be made while remaining within the scope of the various embodiments.

### Example 1

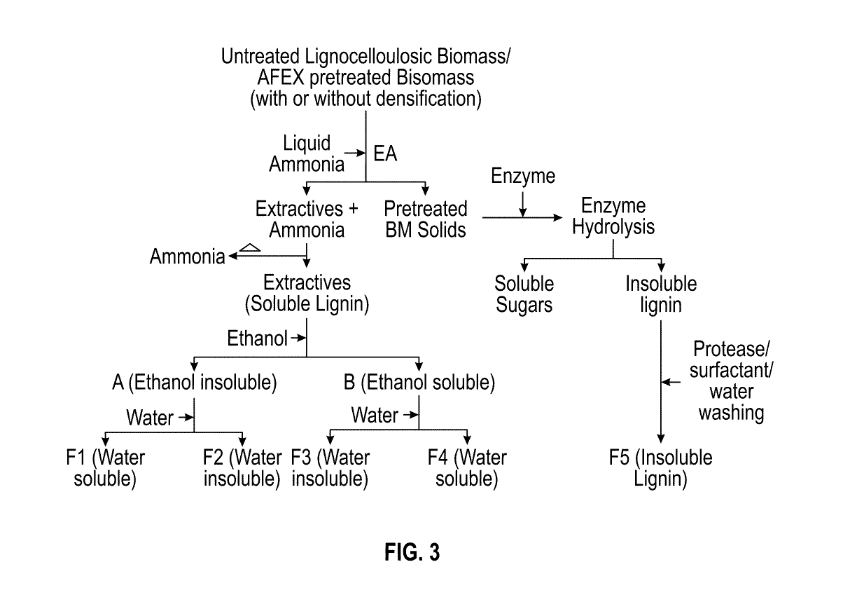

FIG. 3 is a schematic illustration showing the fractions of ammonia-soluble extractives obtained using the EA treatment system, namely F1 (Ethanol insoluble/water soluble), F2 (Ethanol insoluble/water insoluble), F3 (Ethanol soluble and water insoluble), and F4 (Ethanol soluble and water soluble). The extracted biomass was further hydrolyzed using enzymes to obtain insoluble lignin. The lignin was further processed using proteases (Sigma), mild surfactant and water to produce fraction F5 (ammonia-insoluble lignin).

In this testing, corn stover (CS) (Pioneer® 36H56 (triple stack—corn borer/rootworm/Roundup Ready) produced in Arlington Research Station, Wisconsin) having a moisture content (MC) less than 10% was treated using the EA reactor system shown in FIG. 3. The CS was loaded into a custom made tubular reactor, together with additional water to adjust the moisture content to approximately 10%. Ammonia was then loaded and the reaction temperature was increased over a period of 15 min using heating tape (McMaster Carr, USA) and maintained for the desired residence time (RT). Solubilized lignin, together with ammonia, was collected in another tubular vessel by opening a high pressure valve located at the bottom of the tubular reactor. The ammonia was then released from the tubular collection vessel to recover soluble lignin rich extractives in to a beaker.

A variety of AFEX™ pretreatment conditions were used, including, for example, 120° C., 6:1 ammonia to biomass ratio, 10% moisture (based on dry weight of biomass) and 60 minutes RT. For 100 g of biomass (dry weight basis) about 15 g were collected as extractives (5.6 g lignin and 9.4 g other components like carbohydrates, ash etc.). The remaining 85 g of biomass were dried in the hood overnight and stored for enzymatic hydrolysis.

The ammonia-soluble lignin extractives were further fractionated using a combination of solvents such as ethanol and water. (See Figs). First, ethanol was added to the extractives (˜5 times the extract volume) which resulted in ethanol insoluble precipitate (A). This fraction was separated from ethanol soluble fraction (B). Water was added (˜5 times the solution volume) to fraction A to obtain two fractions, namely F1 (water soluble) and F2 (water insoluble). Similarly, water was added to fraction B to get two fractions, namely F3 (water insoluble) and F4 (water soluble). All the samples were lyophilized and the amount of the lignin separated was estimated using National Renewable Energy Lab (NREL) protocol (Table 1).

TABLE 1
Mass balance of fractionated lignin
Total Extractives 15 g
Total lignin 5.6 g

| | F1 | F2 | F3 | F4 |
|---|---|---|---|---|
| Amount fractionated | 0.35 g | 0.06 g | 4.5 g | 1.24 g |
| % of initial lignin in CS | 2.5% | 0.5% | 32.2% | 8.9% |
| % lignin purity | 69.1% | 14.4% | 92.4% | 13.5% |
| Mass Extracted per 100 g | 0.5 g | 0.4 g | 4.9 g | 9.2 g |
| initial CS | | | | |

### Example 2

Corn stover from Idaho National Lab (INL) was pretreated using a PB-AFEX™ pretreatment as described in, for example, U.S. Pat. Nos. 8,394,177, 8,196,533 and 7,937,851, and U.S. patent application Ser. No. 13/458,568.

The resulting corn stover was combined with other starting materials in varying combinations in a tubular packed bed reactor (such as the ones described in the patent publications described herein and assigned to MBI International) and subject to EA under various reaction conditions to produce liquids (soluble lignin, extractives and solvent) and solids (extracted biomass) as shown in Table 2.

After closing the reactor, the reactor and its contents were heated to the desired temperature and held there for a defined RT. Thereafter, the ammonia or solvent soluble extractives, together with the solvent, were removed by purging with high pressure nitrogen. The extractives were collected in a 1 L tubular vessel. The solvent was removed using a Buchi R-210 rotary evaporator (BUCHI, Labortechnik AG, Switzerland) followed by freeze drying to remove water using a high pressure freeze dryer (Labconco 20-port freeze drying unit, Kansas City, Mo.).

The amount of lignin extracted using these solvent combinations was calculated based on the amount of solid recovered after extraction (dry weight basis) as given in Table 2 below.

TABLE 2
Amount of Solids Recovered following Extraction

| Pre- | % Moisture | | | | | Post- | | |
|---|---|---|---|---|---|---|---|---|
| extraction | of PB- | | Solvent to | | | extraction | | |
| biomass | AFEX ™ | Solvent for | biomass | Time | Temp | biomass | % solids | % mass |
| weight (g) | biomass | extraction | ratio (v/v) | (min) | (° C.) | weight (g) | recovered | extracted |
| 75.2 | 10 | Anhyd. | 6 | 30 | 25 | 64 | 85.1% | 14.9% |
| | | NH₃ | | | | | | |
| 75.2 | 10 | Anhyd. | 6 | 30 | 50 | 64.1 | 85.2% | 14.8% |
| | | NH₃ | | | | | | |
| 75.2 | 10 | Anhyd. | 3 | 30 | 50 | 67.5 | 89.8% | 10.2% |
| | | NH₃ | | | | | | |
| 75.2 | 10 | Anhyd. | 3 | 30 | 25 | 65.5 | 87.1% | 12.9% |
| | | NH₃ | | | | | | |
| 75.2 | 10 | 100% | 6 | 30 | 50 | 72.1 | 95.9% | 4.1% |
| | | ETOH | | | | | | |
| 75.2 | 10 | 90% | 6 | 30 | 50 | 70.8 | 94.1% | 5.9% |
| | | Acetone | | | | | | |
| 75.2 | 10 | 30% | 6 | 30 | 50 | 64.6 | 85.9% | 14.1% |
| | | NH4OH | | | | | | |
| 75.2 | 10 | NH₄OH:ETOH = | 6 | 30 | 50 | 64.1 | 85.2% | 14.8% |
| | | 1:1 | | | | | | |
| 75.2 | 10 | 100% | 6 | 30 | 100 | 62.8 | 83.50% | 16.50% |
| | | H₂O | | | | | | |

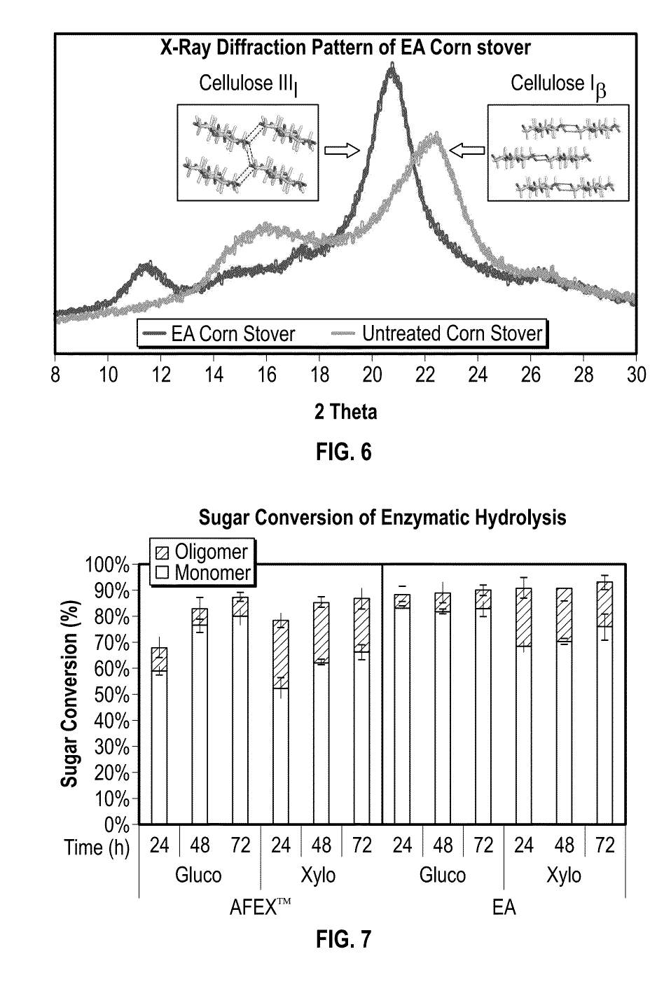

When PB-AFEX™ CS pellets were extracted using anhydrous ammonia at room temperature (25° C.) cellulose III was produced within the biomass. Cellulose III is significantly more digestible by enzymes than native cellulose I biomass present in PB-AFEX™ treated corn stover. (See FIG. 6).

### Example 3

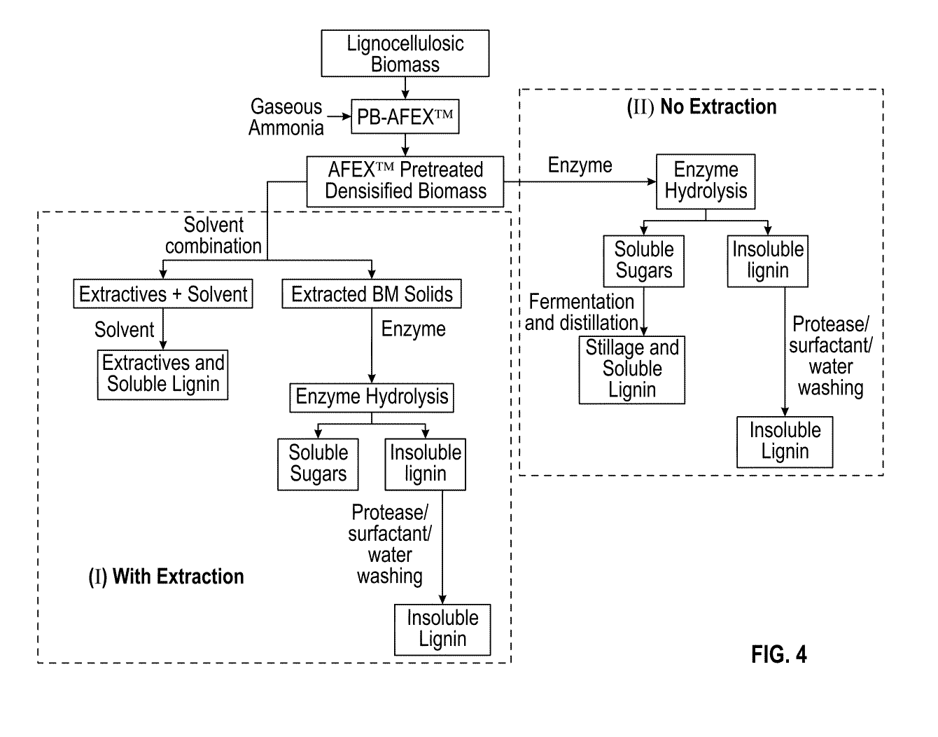

FIG. 4 is a schematic illustration showing two possible scenarios while hydrolyzing PB-AFEX™ densified biomass particulates, namely extraction using (I) anhydrous ammonia or organic solvent or combination of both and (II) no extraction.

In this testing, extracted PB-AFEX™ treated and EA treated corn stover samples were subjected to enzyme hydrolysis at high solid loading (6% glucan loading, w/v).

AFEX™-densified biomass particulates can be hydrolyzed before or after ammonia/solvent based extractions. In Scenario I of FIG. 4, extraction was performed using anhydrous ammonia, an organic solvent or a combination thereof. In scenario II, the hydrolysis is performed without extraction. In this scenario, the water soluble lignin could be recovered as stillage following microbial fermentation.

If extracted (with ammonia/solvents) and then hydrolyzed, a higher sugar conversion rate (due to formation of cellulose III) was obtained. When the biomass was hydrolyzed without ammonia/solvent extraction, a reduced sugar conversion was obtained, due the presence of soluble lignin (and absence of cellulose III).

Hydrolysis experiments were done for a period of 168 h using combination of commercial enzymes Ctec2 and Htec2 (Novozyme) at 30 mg/g of glucan, pH 4.8, 200 rpm, 50° C. After hydrolysis, the soluble sugars were separated from unhydrolyzed solids using centrifugation and decantation and analyzed using a Shimadzu HPLC with Refractive index detector to measure monomeric and oligomeric sugars. The complete mass balance for the sugar conversion and amount of unhydrolyzed solids left behind after hydrolysis at different time points (24, 48, 168 h) a given in Table 3.

TABLE 3
Mass balance after 6% glucan loading hydrolysis of AFEX ™ and
EA treated corn stover at 24, 72 and 168 h.

| | Bio- | Weight of | % | % | Total sugar |
|---|---|---|---|---|---|
| | mass | dry unhydro- | Glucan | Xylan | (Glucan + |
| | added | lyzed solids | conver- | conver- | Xylan) |
| Sample | (g) | (g) | sion | sion | conversion |
| AFEX ™⁻ | 94.84 | 39.12 | 68.17% | 78.82% | 72.24% |
| CS 24 h | | | | | |
| AFEX ™ - | 94.84 | 42.71 | 83.24% | 85.42% | 84.07% |
| CS 72 h | | | | | |
| AFEX ™- | 94.84 | 25.62 | 87.61% | 86.93% | 87.35% |
| CS 168 | | | | | |
| EA-CS 24 h | 83.7 | 17.42 | 88.35% | 90.83% | 89.27% |
| EA-CS 72 h | 83.7 | 16.54 | 89.42% | 90.87% | 89.96% |
| EA-CS 168 h | 83.7 | 19.05 | 90.0% | 93.09% | 91.15% |

The unhydrolyzed solids were further washed with water to remove residual sugars and subjected to composition analysis. The results are given in Table 4.

TABLE 4
Composition of un-hydrolyzed solids (UHS) obtained after enzyme hydrolysis
for AFEX ™ (or PB-AFEX ™) and EA treated corn stover (CS)
samples at 6% glucan loading. AFEX ™-CS-UHS were further subjected
to enzyme hydrolysis and its composition is also given in the table (AFEX ™-CS-UHS-EH).

| | AFEX ™-UHS-CS-6% | | AFEX ™-UHS-CS-EH-6% | | EA-UHS-6% | |
|---|---|---|---|---|---|---|
| Composition | Average | Std dev | Average | Std dev | Average | Std dev |
| Glucan | 24.92% | 0.12% | 16.33% | 0.48% | 18.97% | 0.14% |
| Xylan | 14.80% | 0.18% | 10.21% | 0.26% | 6.54% | 0.04% |
| Arabinan | 1.96% | 0.03% | 1.43% | 0.01% | 1.27% | 0.01% |
| Acetyl | 0.33% | 0.02% | 0.24% | 0.03% | 0.36% | 0.04% |
| Ash | 9.21% | 0.08% | 11.24% | 0.17% | 12.98% | 0.11% |
| Acid insoluble lignin | 37.79% | 0.32% | 49.72% | 0.46% | 39.92% | 0.48% |
| Acid soluble lignin | 3.09% | 0.07% | 2.64% | 0.19% | 4.05% | 0.11% |

The composition analysis reveals that un-hydrolyzed solids contain a considerable amount of glucan and xylan. Therefore, enzymatic hydrolysis was performed on the AFEX™-CS-UHS for another 72 hours and found a considerable drop in the glucan and xylan content. This process was repeated several times to isolate insoluble lignin from the unhydrolyzed solids. A considerable amount of cellulase and hemicellulase enzymes were bound to the substrate after the hydrolysis reaction. In order to further purify the insoluble lignin proteases were used to digest the enzymes by incubating the insoluble lignin fraction (F5) overnight at 37° C. in phosphate buffer (30 ml, 20 mM, pH 7), containing 5 mg protease (Sigma). The remaining protease was deactivated by incubation at 90° C. for 2 h. The residue was washed extensively with phosphate buffer and distilled water and freeze-dried with the Labconco freeze dryer.

### Example 4

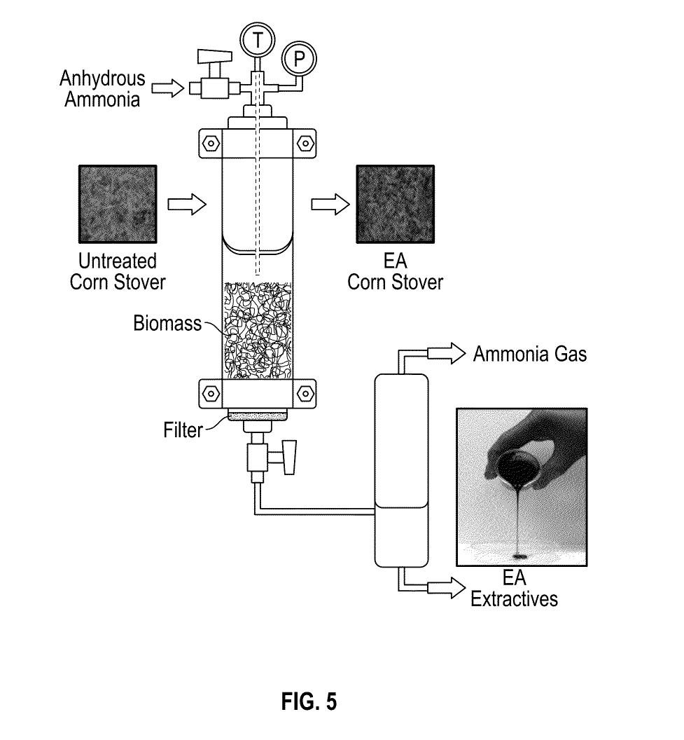

FIG. 5 is a schematic illustration of EA treatment process flow diagram containing images of “untreated corn stover” and “EA” treated corn stover, and the resulting EA extractives.

These tests were run using biomass having a 10% moisture content (MC) wet-based, an ammonia to biomass ratio of 6:1, a temperature of about 120° C., and residence time (RT) of 30 minutes.

Enzymes used included: Cellic® CTec2=50% (protein basis), Cellic® HTec2=25% (protein basis) and Multifect Pectinase=25% (Protein basis). The total enzyme loading was 15 mg/g glucan.

In this example, multiple fractions generated from extractives of EA treated corn stover were characterized using several techniques, including GPC, ¹³C-NMR, ³¹P-NMR and 2D NMR. Additionally, the level of lignin extraction and cellulose III conversion was correlated with cellulose digestibility for corn stover.

### Untreated Corn Stover

Corn stover (Pioneer 36H56) was harvested in September 2009 in Wisconsin (USA) and oven dried at 60° C. for approximately 2 weeks. The biomass was further passed through a 5 mm screen installed in a Christy hammer mill (Christison Scientific LTD, England) and stored at 4° C. in heat sealed bags prior to utilization. The moisture content of the dried and milled corn stover was approximately 6% in a wet weight basis. The biomass composition analysis was performed using NREL protocols NREL/TP-510-42618 and NREL/TP-510-42620. On a dry weight basis, the untreated corn stover contained approximately 35% glucan, 21% xylan, 1% galactan, 3% arabinan, 14% lignin, 5% ash and 15% extractives (i.e. ethanol and water soluble compounds).

### EA Treatment

EA treatment was conducted in 1 Liter (L) high pressure, stainless steel tubular reactors custom built using components purchased from McMaster Car Company, USA and equipped with individual heating mantles, temperature and pressure indicators. The reactors were connected to the temperature controlled, high pressure 1 L collection vessel to allow separation of ammonia from the extractives by evaporation. In each reactor, 40 grams of corn stover (dry weight basis), containing 10% moisture (wet weight basis) reacted with 240 grams of ammonia for 30 minutes at 120° C. Set point temperature was achieved in the first 7 minutes of reaction, allowing the pressure to reach 83 bar. The pressure in the flash tank was equilibrated with nitrogen to the same pressure observed in the reactor during pretreatment.

Upon reaction completion, the bottom valve in the reactor was open, allowing liquid ammonia and soluble extractives to pass through a stainless steel filter of 50 μm pore size. The ammonia gas was then released from the top of the flash tank by opening a valve, while pressure was maintained in the reactor at 83 bar using nitrogen for 10 minutes. This procedure was designed to maintain ammonia in the liquid state during extraction and allow residual ammonia to be efficiently released from the biomass. After extraction, the reactor and flash tank pressures were equalized to ambient pressure by releasing the ammonia and nitrogen gas from the top of the flash tank. During evaporation, the liquid extractives precipitated in the bottom of the flash tank.

The biomass was then removed from the tubular reactors and allowed to dry overnight in the hood. The system lines were further cleaned with 70% ethanol and 90% acetone (Research Grade, BD Chemicals) to remove residual extractives from the lines, which were collected in the flash tank. All the EA extractives were then drained from the flash tank, collected in a 1 L round bottom flask and further concentrated in vacuum using the rotary evaporator (BUCHI, Switzerland) set at 70° C. The EA extractives were then dried with the Labconco freeze dryer. The dry weight of the extractives and EA treated biomass was further recorded for mass balance purposes. The dried samples were stored at 4° C. in sealed containers to avoid major moisture exposure.

EA Extractives Fractionation and Production of Lignin Rich Streams from EA Process

Freeze-dried EA extractives were solubilized in 100% ethanol (Research Grade, BD Chemicals) using 1:10 (w/v) extractives-to-solvent ratio for 30 minutes at continuous mixing conditions with a glass rod. The ethanol insoluble fraction was filtered using a fibreglass filter installed in a Millipore vacuum filter holder (EMD Millipore, Billerica, Mass., USA). The filtrate was further washed with fresh 100% ethanol to remove residual ethanol-soluble components adsorbed to the solid fraction. The solid fraction was air dried for 2 hours in the hood to evaporate residual ethanol in the sample. The dried ethanol insoluble sample was weighed and placed in a beaker containing distilled water in a 1:10 (w/v) extractives-to-water ratio and stirred for 30 minutes under mixing conditions. The resulting suspension was vacuum filtered using a fiberglass filter and the filtrate washed with water to remove residual water soluble components.

The water insoluble fraction resulting from this separation was transferred to a pre-weighed 50 ml plastic Falcon tube, and dried using the freeze dryer. The dried sample was weighed for mass balance purposes and labelled as Fraction 1 (F1). The water soluble fraction was collected in a 1 L round bottom flask and concentrated using the rotary evaporator (BUCHI, Switzerland) under vacuum, while allowing it to dry. The sample was further transferred to a pre-weighed container and freeze dried with the freeze dryer. The dried sample was weighed for mass balance purposes and labelled as Fraction 2 (F2).

The ethanol soluble fraction was transferred to a pre-weighed round bottom flask and dried using the rotary evaporator at 60° C. under vacuum. Distilled water in a 1:10 (w/v) extractives-to-water ratio was added to the dried ethanol soluble fraction and mixed for 30 minutes to solubilise water soluble extractives. The suspension was vacuum-filtered and washed with distilled water using a membrane placed on top of a sintered silica funnel to remove additional water soluble components adsorbed to the water insoluble fraction. The water insoluble fraction was transferred to a pre-weighed container and freeze dried. The dry weight of the sample was recorded for mass balance purposes and labelled as Fraction 3 (F3).

The filtered water soluble fraction was further concentrated using the rotary evaporator under vacuum at 80° C., while avoiding to reach dryness. The concentrated fraction was transferred to a pre-weighed container and freeze dried. The dried sample was weighed for mass balance purposes and labelled as Fraction 4 (F4). All freeze dried samples were stored in the fridge at 4° C. in sealed plastic containers prior to utilization to avoid moisture exposure.

### Enzymatic Hydrolysis (EH) of EA Treated Corn Stover

Enzymatic hydrolysis (EH) was performed at 6% glucan loading, using 15 mg of enzyme per gram of glucan in a 5 Litre bioreactor set to control mixing speed at 120 RPM, temperature of 50° C. and pH 4.8 for 72 hours. The enzymes utilized in this work were Cellic® CTec2 (138 mg protein/ml, batch No. VCNI0001) and Cellic® HTec2 (157 mg protein/ml, batch No. VHN00001) provided by Novozymes® (Franklinton, N.C., USA). The enzymatic cocktail was also supplemented with Multifect Pectinase (MP) (72 mg protein/ml, batch No. 4861295753), from Genencor (Pala Alto, Calif., USA). The protein concentration for the enzymes was determined using the Kjeldahl nitrogen analysis method (AOAC Method 2001.11, Dairy One Cooperative Inc., Ithaca, N.Y., USA). The enzyme ratios utilized in this work was 50% Cellic® CTec2, 25% Cellic® HTec2 and 25% MP in a dry protein weight basis.

After EH, the resulting suspension was centrifuged at 8,000 RPM for 30 minutes in a Beckman Coulter Avanti J-26XP centrifuge, equipped with a rotor model JLA 8.1000 (Beckman Coulter, Inc., Brea, Calif., USA), to separate the unhydrolyzed solids (UHS) from the liquid hydrolyzate. The liquid hydrolyzate was decanted to a volumetric cylinder and the volume was recorded for mass balance purposes. The UHS were washed twice with equivalent volume of distilled water as of hydrolyzate. This washing step was performed by sequential re-suspension of the solids, centrifugation and decantation. The solution resulting from the washing steps was transferred to a volumetric cylinder and the volumes were recorded for mass balance purposes. Samples of the hydrolyzate and water washing solutions were prepared for glucose and xylose analysis using HPLC equipped with a Bio-Rad Aminex HPX-87H column (Bio-Rad, Hercules, Calif., USA) as previously described. (See FIG. 7).

### Lignin Mass Balance

Mass balance on lignin was performed around the EA treatment, EA extractives fractionation and enzymatic hydrolysis. The dry weight loss observed during pretreatment of corn stover was calculated by measuring the weight of the biomass before and after pretreatment, along with the moisture content using a moisture analyzer A&D MX-50 (A&D Engineering, Inc., San Jose, Calif., USA). Composition analysis was performed on corn stover before and after EA treatment using the standard NREL protocols NREL/TP-510-42618 and NREL/TP-510-42620. Nitrogen analysis was performed using a nitrogen analyzer.

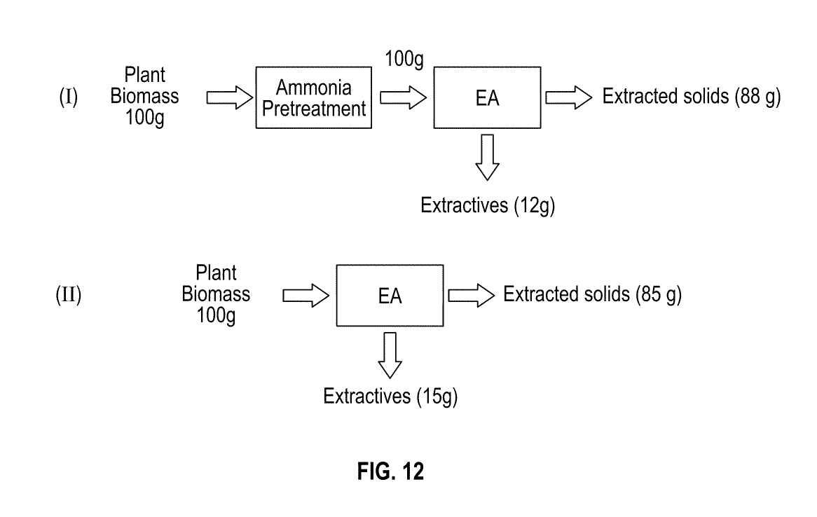

Lignin extraction yield was calculated by the difference in total lignin weight before and after pretreatment, divided by the total lignin weight of the untreated sample. The EA extractives fractionation mass balance over lignin was performed by measuring the dry weight of each fraction and performing composition analysis following the NREL protocols mentioned above. The percent recovery of each fraction was normalized with respect to the total lignin present in corn stover and total extracted lignin during EA treatment. (See, for example, FIG. 12).

EA treatment on untreated corn stover. Pretreatment condition include: 120° C. using 3:1 ammonia:biomass ratio for 30 minutes residence time.

FIG. 8 is a flow chart showing EA fractionation and lignin mass balance according to various embodiments. Compositional analysis of Fractions F1-F5 is shown in Table 5.

TABLE 5
Compositional analysis of Fractions F1-F5

| | F1 | F2 | F3 | F4 | F5 |
|---|---|---|---|---|---|
| Glucan | 0.45% | 13.29% | 0.04% | 4.43% | 18.97% |
| Xylan | 0.16% | 11.72% | 0.01% | 1.28% | 5.54% |
| Arabinan | 0.03% | 3.21% | 0.00% | 0.53% | 1.27% |
| Acetyl | 0.39% | 1.24% | 0.35% | 27.28% | 0.36% |
| Lignin | 69.12% | 14.44% | 92.35% | 13.49% | 42.9% |
| Ash | 1.43% | 4.01% | 0.18% | 2.35% | 4.05% |

### Characterization of EA Extractives Fractions

### Lignin Characterization by NMR

2D-HSQC NMR Analysis

Nuclear magnetic resonance (NMR) spectra of samples in DMSO-d6/pyridine-d5 (4:1, v/v) were acquired by using a Bruker Biospin (Billerica, Mass., USA) AVANCE 500 (500 MHz) spectrometer fitted with a cryogenically-cooled 5 mm TCI gradient probe with inverse geometry (proton coils closest to the sample) and spectral processing used Bruker's Topspin 3.1 (Mac) software. The central DMSO solvent peaks were used as internal reference (δ_{H}/δ_{C}: 2.50/39.51).

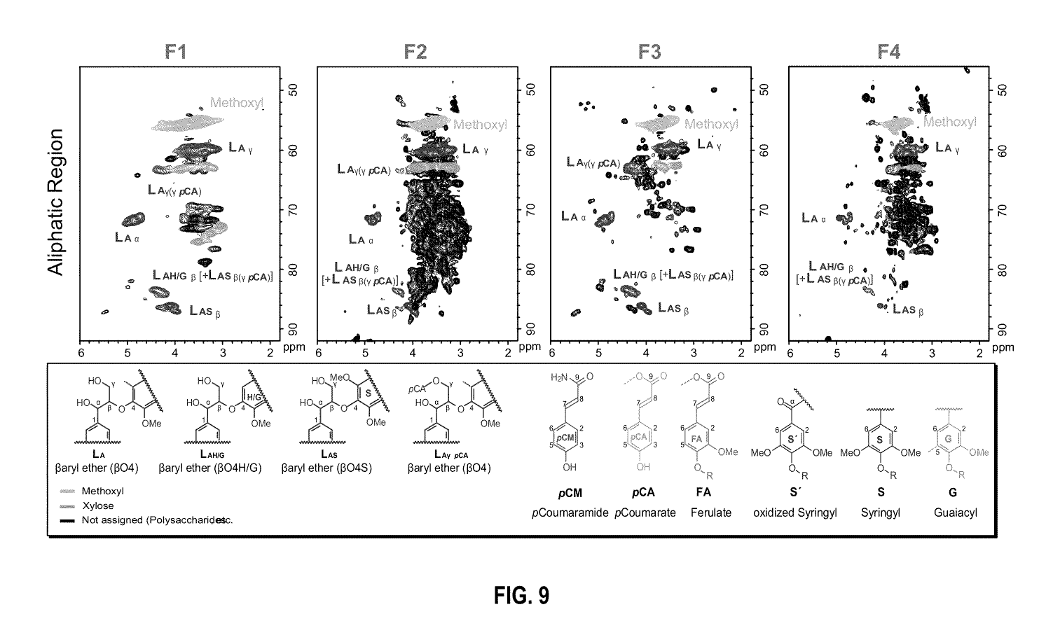

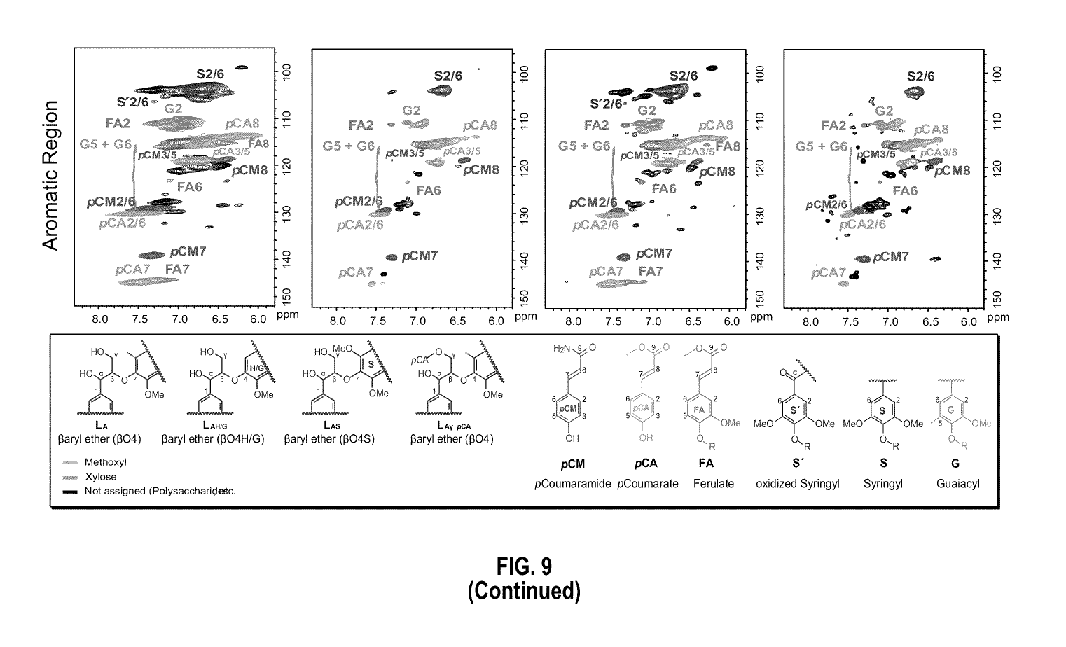

FIG. 9 show 2D HSQC-NMR (in DMSO-d6) of fractions F1-F4, with components identified in the aliphatic and aromatic regions.

### ¹³C-NMR and ³¹P-NMR Analysis

Lignin was isolated from corn stover and UHS by ball milling and organic-solvent extraction. Ball milled lignin (BML) samples of corn stover and UHS were prepared according to known procedures. Lignin isolation of EA extractive fractions was performed according to the same procedures as corn stover and UHS, excluding ball milling.

Isolated lignin samples (˜100 mg) were dissolved in DMSO-d6 (500 mg) and analyzed by quantitative ¹³C NMR using a Bruker Avance-400 MHz spectrometer at a frequency of 100.59 MHz with an inverse gated decoupling pulse sequence using a 12 s pulse delay and 10K scans. Quantitative ³¹P NMR analysis of BML (˜25 mg) was accomplished by using a pyridine/CDCl3 (1.6:1, v/v) solvent, cyclohexanol as an internal standard and 2-chloro-4,4,5,5-tetramethyl-1,3,2-dioxaphospholane (TMDP) as the derivatization agent following the method described in Granata A, Argyropoulos DS (1995), 2-Chloro-4,4,5,5-tetramethyl-1,3,2-dioxaphospholane, a reagent for the accurate determination of the uncondensed and condensed phenolic moieties in lignin, J Agric Food Chem 43 (6):1538-1544

The ³¹P NMR spectra were acquired using an inverse gated decoupling pulse sequence with a 25 sec pulse delay and 128 scans.

Table 6 shows an abundance of assigned hydroxyl groups of fractionated EA extractives from corn stover, determined by ³¹P NMR.

TABLE 6
Assigned hydroxyl groups of fractionated EA extractives
from corn stover, determined by ³¹P NMR

| | Percentage of total OH | | | | |
|---|---|---|---|---|---|
| Assignments | F1 | F2 | F3 | F4 | F5 |
| Aliphatic-OH | 61.89% | 84.28% | 47.28% | 51.53% | 73.79% |
| C-5 + C-3 | 6.82% | 2.99% | 8.17% | 1.37% | 6.85% |
| substituted-OH | | | | | |
| C-3 substituted/p- | 22.39% | 4.38% | 25.96% | 10.51% | 11.57% |
| coumaryl/p-hydroxy- | | | | | |
| phenyl-OH | | | | | |
| Carboxylic acid-OH | 8.90% | 8.34% | 18.59% | 36.59% | 7.79% |
| Total (mmol —OH/g | 5.08 | 9.39 | 4.94 | 11.60 | 4.87 |
| Lignin) | | | | | |

Table 7 shows assignments and integration values of quantitative ¹³C NMR spectra of fractionated EA extractives from corn stover.

TABLE 7
Assignments and integration value of quantitative ¹³C NMR
spectra of fractionated EA extractives from corn stover

| Chemical | | Amount/Ar | | | | |
|---|---|---|---|---|---|---|
| Shift (ppm) | Assignments | F1 | F2 | F3 | F4 | F5 |
| 184-180 | C═O in | 0.08 | 0.13 | 0.04 | 0.00 | 0.07 |
| | spirodienone unit | | | | | |
| 176-169 | Aliphatic COOR | 0.40 | 2.33 | 0.42 | 10.64 | 1.03 |
| 169-166 | Conjugated COOR | 0.30 | 0.41 | 0.27 | 1.43 | 0.27 |
| 163-157 | PCA₄ | 0.25 | 0.37 | 0.21 | 0.90 | 0.19 |
| 156-142 | C₃/C′₃ in 5-5′ biphenyl | 1.79 | 1.37 | 1.60 | 1.57 | 1.98 |
| | and C₃/C₄ in G units, | | | | | |
| | C₃/C₅ in S units | | | | | |
| 142-124 | C₁ G units, C₅/C₅′ in | 1.97 | 1.45 | 2.42 | 2.84 | 2.07 |
| | etherified 5-5 units | | | | | |
| 123-117 | C₆ in G units | 0.34 | 0.17 | 0.28 | 0.88 | 0.38 |
| 117-113 | C₅ in G units | 0.96 | 0.72 | 1.01 | 2.48 | 0.72 |
| 113-109 | C₂ in G units | 0.15 | 0.14 | 0.20 | 0.20 | 0.17 |
| 108-103 | C₂/C₆ in S units | 1.11 | 2.45 | 0.80 | 0.01 | 1.02 |
| 90-78 | C_{β} in β-O-4, Cα in | 0.84 | 2.84 | 0.50 | 0.07 | 0.51 |
| | β-5 and β-β | | | | | |
| 65-62 | C_{γ} in β-5 and β-O-4 | 1.28 | 0.98 | 1.31 | 0.91 | 1.23 |
| | with α-carbonyl | | | | | |
| | structures (G and S | | | | | |
| | units) | | | | | |
| 61-57 | C_{γ} in β-O-4 without α | 0.26 | 1.57 | 0.17 | 3.24 | 0.11 |
| | carbonyl structures | | | | | |
| | (G and S units) | | | | | |
| 58-55 | Methoxy CH₃ | 0.66 | 2.35 | 0.36 | 0.58 | 0.56 |
| 53-51 | C_{β} in β-β and β-5 | 0.02 | 0.06 | 0.04 | 0.15 | 0.04 |
| | structures | | | | | |
| 35-22 & | Aliphatic Carbons | 0.72 | 0.79 | 3.68 | 1.68 | — |
| 20-10 | | | | | | |
| 22-20 | Acetyl CH₃ | 0.00 | 0.49 | 0.36 | 12.71 | — |
| S/G ratio | (I₁₀₈₋₁₀₃/2)/I₁₁₄₋₁₀₉ | 3.65 | 8.62 | 1.97 | 0.02 | 2.92 |

Residual Carbohydrates: 105-95 & 89-60 ppm
PCA: para-coumaric acid; G: Guaiacyl; S: Syringyl

### Gel Permeation Chromatography (GPC)

The isolated lignin samples (100 mg) were treated with a mixture of pyridine and acetic anhydride (1:1, v/v, 4 ml) with stirring at room temperature for 24-36 h. The reaction mixture was diluted with ethanol (30 mL) and stirred for 30 min and then concentrated under lower pressure. The acetylated lignin samples were dissolved in chloroform (2 mL) and added dropwise into diethyl ether (100 mL) to precipitate the sample followed by centrifugation. The precipitate was washed with diethyl ether and centrifuged three times. After air drying, the acetylated samples were dried for 24 h in a vacuum oven at 40° C. prior to GPC analysis.

Molecular weight determination was conducted using a Polymer Standards Service (PSS) GPC Security 1200 system equipped with four Waters Styragel columns (HR0.5, HR2, HR4, HR6) at 30° C., Agilent isocratic pump, Agilent auto-sampler, Agilent degasser, Agilent refractive index (RI) detector and Agilent UV detector (270 nm) using THF as the mobile phase (1 mL/min) with injection volumes of 20 μL.

The weight average molecular weight (M_{w}) of the derivatized lignin samples were acquired by using an relative calibration curve and this relative calibration curve was created by fitting a third order polynomial equation to the retention volumes obtained from a series of narrow molecular weight distribution polystyrene standards (1.36×10⁶, 5.38×10⁵, 3.14×10⁴, 7.21×10³, 4.43×10³, 5.80×10² g/mol). The curve fit had an R² value of 0.9984.

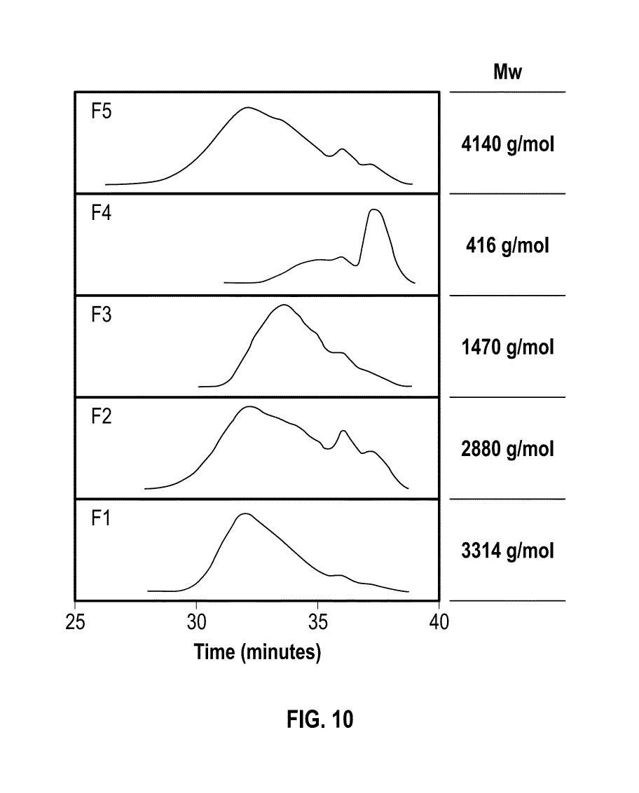

FIG. 10 are gel permeation chromatography graphs of fractions F1-F5 according to various embodiments.

### Elemental Analysis

The inorganic elements in the samples were determined by Inductively Coupled Plasma Emission Spectroscopy (ICP) according to methodology previously employed by Allison et al. J (2000) Metal profiling of southeastern U.S. softwood and hardwood Furnish, Tappi J 83 (8):97-10. Nitrogen, Carbon, Hydrogen and Oxygen analysis in fraction F3 were performed by Galbraith Laboratories, Inc., Knoxyille, Tenn., USA

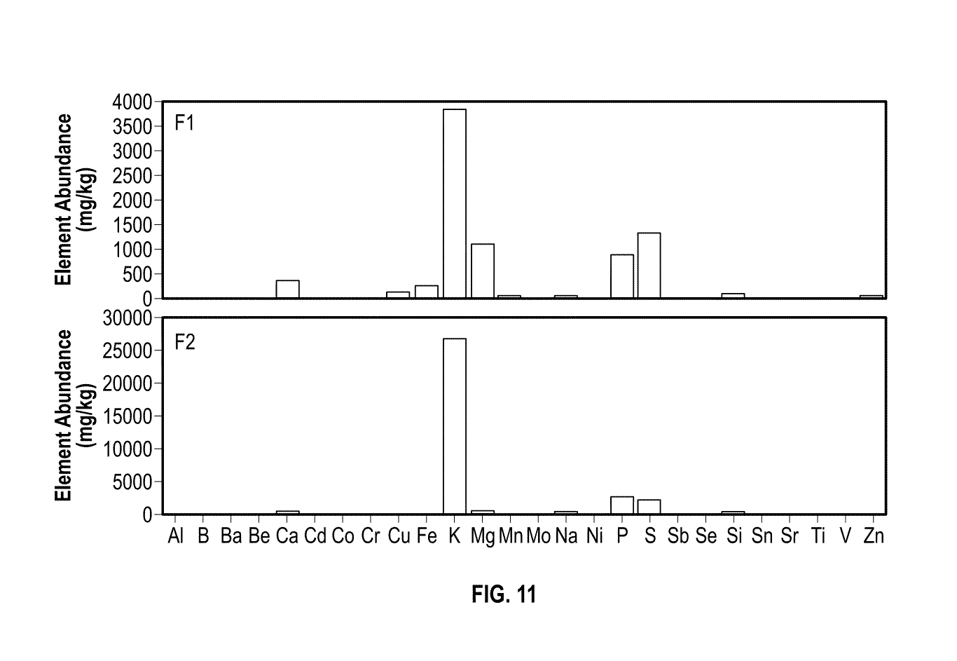

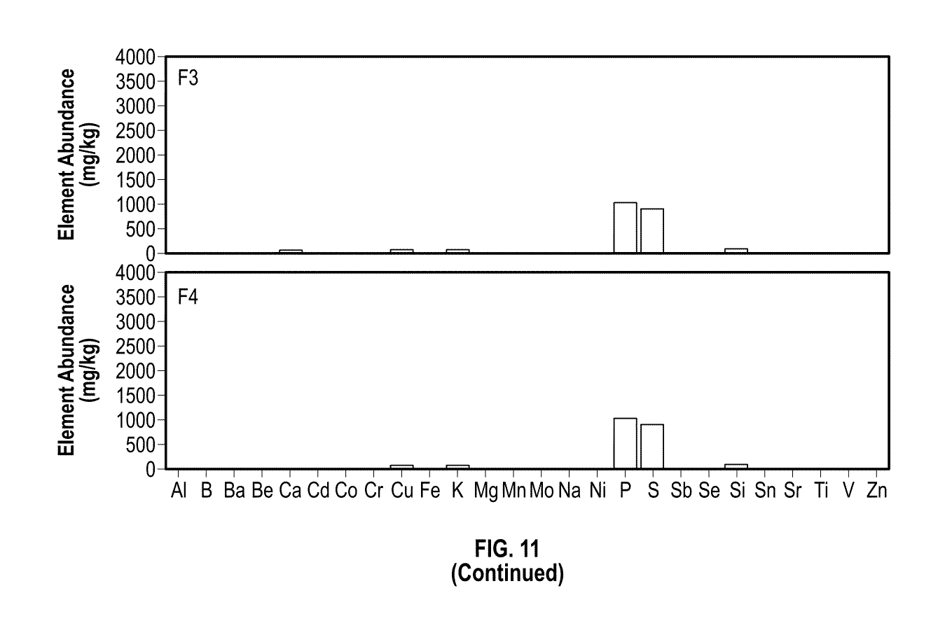

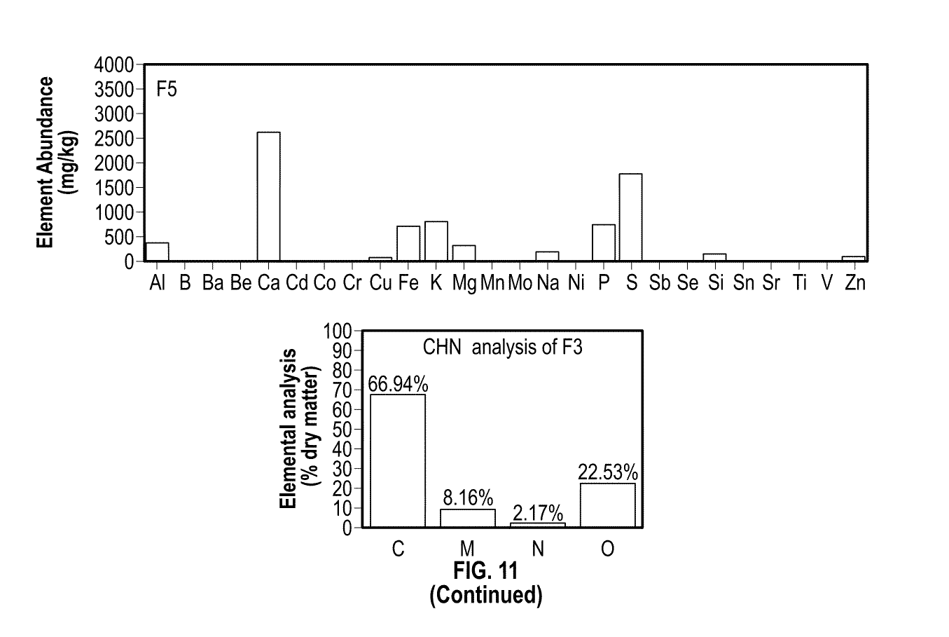

FIG. 11 show elemental analysis graphs of fractions F1-F5, including a more detailed elemental analysis graph of F3. Table 8 shows elemental compositions of F1-F5.

TABLE 8
Composition (mg/kg)

| Element | F1 | F2 | F3 | F4 | F5 |
|---|---|---|---|---|---|
| Al | 12.5 | 11.7 | 3.0 | 2.1 | 390.1 |
| B | 0.4 | 11.4 | <0.3 | <0.3 | 3.2 |
| Ba | 2.5 | 3.3 | 1.3 | 1.0 | 29.0 |
| Be | <0.0 | <0.0 | <0.0 | <0.0 | <0.0 |
| Ca | 361.7 | 288.6 | 27.6 | 24.5 | 2714.0 |
| Cd | <0.5 | <0.5 | <0.5 | <0.4 | <0.5 |
| Co | <0.7 | <0.7 | 0.7 | 1.0 | <0.7 |
| Cr | 4.8 | 2.7 | 0.9 | 4.6 | 14.6 |
| Cu | 134.0 | 36.6 | 33.2 | 51.7 | 59.2 |
| Fe | 248.5 | 63.6 | 11.5 | 17.5 | 750.3 |
| K | 3830.0 | 26643.4 | 131.2 | 75.6 | 845.9 |
| Mg | 1120.0 | 496.8 | 11.2 | 7.5 | 345.3 |
| Mn | 34.6 | 20.9 | 2.1 | 1.0 | 28.7 |
| Mo | <1.6 | 4.3 | 1.6 | 3.2 | 3.0 |
| Na | 40.3 | 388.5 | 39.6 | 12.3 | 187.9 |
| Ni | 3.7 | 2.5 | 1.5 | 10.2 | 9.8 |
| P | 898.9 | 2573.3 | 209.7 | 1033.1 | 754.9 |
| S | 1340.0 | 2305.0 | 1310.0 | 911.6 | 1860.0 |
| Sb | <5.1 | <5.1 | <5.1 | <4.6 | <5.1 |
| Se | <11.3 | <11.3 | <11.3 | <10.1 | <11.4 |
| Si | 100.9 | 242.9 | 53.5 | 100.0 | 162.6 |
| Sn | 6.3 | 8.3 | 7.4 | 6.5 | 4.7 |
| Sr | 1.2 | 1.0 | 0.1 | 0.1 | 7.4 |
| Ti | 0.8 | 1.4 | 0.5 | 0.1 | 13.2 |
| V | <0.2 | <0.2 | <0.2 | <0.2 | 1.6 |
| Zn | 62.5 | 59.7 | 30.1 | 16.5 | 99.1 |
| C | ND^{a} | ND | 67.0% | ND | ND |
| H | ND | ND | 8.2% | ND | ND |
| N | 3.67% | 2.74% | 2.3% | 13.24 | ND |
| O | ND | ND | 22.5% | ND | ND |

### CONCLUSION

In the above testing, the EA treatment was able to extract about 44% of the lignin present in the corn stover using relatively mild conditions (120° C., 30 min., 6:1 NH₃: CS loading). F3 comprises about 92% lignin, and about 32% of the lignin extracted from the plant biomass is from fraction F3.

NMR data showed that β-aryl ether bonds from lignin were preserved during EA treatment. This is in contrast to conventional lignin processes which use much harsher conditions and can cleave these bonds. NMR also suggests that ferulic and coumaric contents are majorly present in the water soluble and ethanol soluble fraction (F4).

³¹P NMR data showed that the aliphatic —OH groups are the most abundant in all the fractions, with F4 contains the highest density of —OH groups and F5 containing the lowest (See FIG. 9). F4 has aliphatic and aromatic acids (acetic acid, acetamide, ferulic acid, ferulamide, coumaric acid and coumaryl amide. As noted herein, Fraction F4 may be useful as polymer precursors.

F3 has low mineral content (i.e., less than 0.18 wt %) and, as noted herein, shows potential for further processing of lignin to biofuels and biochemicals through catalytic conversion.

### Example 5

Thermal gravimetric analysis (TGA) was done to understand the stability of the lignin fractions under varying temperature conditions. The data was obtained using PerkinElmer Simultaneous Thermal Analyzer (STA) 6000. Samples were put into a ceramic crucible with a lid. The ramp rate was 10° C. per minute from about 25 to about 600° C. When the temperature reached 600° C., it was held under isothermal condition for approximately two minutes. Nitrogen was used as the flushing gas set at 50 mL/min throughout the test. The stability of the fraction depended on the composition of the lignin. Fraction F3 (defined above) was found to be relatively stable, due to its very low carbohydrate content.

Differential scanning calorimeter (DSC) data was collected to understand the stability of lignin fractions under varying temperature conditions. DSC data was obtained using a Q20 TA Instruments V24.7 Build 119. Samples were put into an aluminum pan with a lid and underwent a heat-cool-heat cycle at a ramp rate of 10° C. min−1, including 25 to 400° C., 400 to 100° C. and 100 to 400° C. As a reference, an identical empty aluminum pan with a lid was used. Nitrogen was used as the flushing gas set at 50 mL/min. The glass transition temperature for F3 was about 120° C., which was lower than expected.

In one embodiment, an extractive ammonia (EA) treatment can be used to simultaneously convert cellulose I to cellulose III and partially remove lignin from plant biomass. The resultant pretreated feedstocks contain highly digestible carbohydrate content and a significantly reduced lignin content.

See, for example, FIG. 12, in which an EA process is performed on pretreated plant biomass. In one embodiment, the EA process is an anhydrous ammonia based cellulose III activation process and the pretreated plant biomass is AFEX™ pretreated plant biomass, such as, for example, densified pretreated plant biomass, such as, for example, loose or densified pretreated corn stover (CS). In one embodiment, activation can be performed at a temperature ranging between about 22 and about 28 C, such as at least about 25° C. using a suitable ammonia to biomass loading, such as up to 3:1 for a suitable residence time (RT) of between about 20 and 40 min, such as no more than about 30 min. Temperature, ammonia concentration and solvent type are the major factors that contribute to a selective lignin solubilization and removal. As such, the extracted yield is variable, depending on these and other factors, such as ammonia loading and RT.

Depending on the above mentioned pretreatment conditions, it is also possible to observe variations on the selectivity during lignin solubilization. Some pretreatment conditions tend to solubilize ash and hemicellulose residues along with lignin, while other conditions selectively remove lignin with significantly less carbohydrates.

Although specific embodiments have been illustrated and described herein, it will be appreciated by those of ordinary skill in the art that any procedure that is calculated to achieve the same purpose may be substituted for the specific embodiments shown. For example, although the process has been discussed using particular types of plant biomass, any type of plant biomass, such as grasses, rice straw and the like, for example, may be used. This application is intended to cover any adaptations or variations of the present subject matter. Therefore, it is manifestly intended that embodiments of this invention be limited only by the claims and the equivalents thereof.

What is claimed is:

1. A method comprising:
converting at least a portion of native cellulose I_{β} to cellulose III_{I} by treating plant biomass with liquid ammonia and/or one or more organic solvents to produce a treated plant biomass containing lignin and a lignin fraction; and
extracting lignin and/or other plant cell wall components from the lignin fraction to produce a lignin extract capable of being fractionated.

2. The method of claim 1 wherein substantially all of the native cellulose I_{β} is converted to cellulose III_{I}, further wherein β-aryl ether bonds in the lignin are preserved.

3. The method of claim 1 wherein the other cell wall components are selected from hemicellulose, arabinan, and combinations and degradation products thereof.

4. The method of claim 1 wherein the plant biomass is a monocot or dicot.

5. The method of claim 1 wherein the extracting step is a pretreatment step.

6. The method of claim 5 wherein the plant biomass comprises untreated plant biomass.

7. The method of claim 6 wherein the extracting step is performed simultaneously with a pretreatment step.

8. The method of claim 7 wherein the pretreatment step comprises liquid ammonia fiber expansion (AFEX) pretreatment or gaseous AFEX pretreatment.

9. The method of claim 8 wherein the gaseous AFEX pretreatment step is a packed bed-AFEX (PB-AFEX) pretreatment step.

10. The method of claim 1 wherein the plant biomass comprises one or more densified biomass particulates.

11. The method of claim 1 wherein the liquid ammonia is selected from anhydrous ammonia, concentrated ammonia, dilute ammonium hydroxide and combinations thereof an the one or more organic solvents are selected from an alcohol, a ketone, an ester, and aromatic and any combinations thereof.

12. A product made according the process of claim 1.

13. The method of claim 1 wherein the extracted lignin and/or other plant cell wall components are converted to resins, polymers, biofuels, biochemicals, heat and/or electricity.

14. The method of claim 1 wherein the method further comprises fractionating at least a portion of the lignin extract into four individual fractions based on differences in solubility in one or more polar or non-polar solvents and water.

15. The method of claim 1 wherein said polar solvents comprise water, alcohol and combinations thereof and said non-polar solvents comprise hexane, toluene and combinations thereof.

16. The method of claim 14 wherein the four individual fractions comprise an alcohol insoluble/water soluble fraction (F1), an alcohol insoluble/water insoluble fraction (F2), an alcohol soluble and water insoluble fraction (F3) and an alcohol soluble and water soluble fraction (F4).

17. The method of claim 16 wherein the alcohol is ethanol.

18. The method of claim 16 wherein F1 contains primarily minerals and F2 contains primarily oligomeric sugars, wherein F1 and F2 are both useful as a fertilizer.

19. The method of claim 16 wherein up to about 44% of the lignin in the plant biomass is extracted.

20. The method of claim 19 wherein F3 comprises at least 92% lignin and up to 32% of the lignin extracted from the plant biomass is from fraction F3.

21. The method of claim 16 wherein F3 has a mineral content less than 0.18% by weight and is useful in catalytic transformation using hydrogenation reactions.

22. The method of claim 16 wherein F3 contains primarily low molecular weight lignin components which are useful as antimicrobial agents in paints, adhesive binders and coatings.

23. The method of claim 22 further comprising reducing the low molecular weight lignin components using catalytic hydrogenation.

24. The method of claim 16 wherein F4 contains primarily lignin degraded products together with oligomeric sugars, wherein F4 is useful as a polymer precursor.

25. The method of claim 1 wherein the treated plant biomass is hydrolyzed to produce an insoluble lignin fraction (F5) and a hydrolyzed monomeric sugar fraction containing lignin (F6).

26. The method of claim 25 wherein fraction F5 is further processed to produce substantially pure lignin.

27. The method of claim 26 wherein the substantially pure lignin is useful in carbon fiber production or energy generation.

28. A product made according to the process of claim 14.

29. A reactor system comprising:
a reactor adapted to convert native cellulose I_{β} to cellulose III_{I} by treating plant biomass with liquid ammonia and/or one or more organic solvents to produce a treated plant biomass containing lignin and a lignin fraction and extract lignin and/or other plant cell wall components from the lignin fraction to produce a lignin extract; and
a monitoring system to monitor the reactor.
